(19) United States
(12) Patent Application Publication
(10) Pub. No.: US 2009/0263385 A1
(43) Pub. Date: Oct. 22, 2009

(54) Breast carcinoma treatment method

(76) Inventor: Thadeus J. Schulz, Roswell, GA (US)

Correspondence Address: DAVID PRASHKER P.C., P.O.Box 5387, Magnolia, MA, 01930-5209, US

(21) Appl. No.: 12/383,320

(22) Filed: Mar. 23, 2009

Related U.S. Application Data
(60) Provisional application No. 61/124,621, filed on Apr. 18, 2008.

(51) Int. Cl. A61K 39/395

(52) U.S. Cl. 424/133.1; 424/130.1; 424/172.1

(57) ABSTRACT

The instant invention is an in-vivo treatment method directed against living epithelial cells in the human breast, and uses a modified ductal lavage technique to infuse a purposely formulated treatment fluid into pre-chosen individual ducts in the human breast for reaction with such epithelial cells as then are present within the ducts. The prepared treatment fluid, at a minimum, comprises: at least one preselected Complement-fixing antibody (or preferably an admixture of different Complement-fixing antibodies) directed at specific antigens, haptens or epitopes which are characteristically present on normal, atypical, or malignant breast epithelial cells in-vivo; the recognized chemical components for activating and fixing Complement in-situ; and a biocompatible fluid carrier. The treatment method can be employed as a therapeutic process in-vivo against a presently existing breast disorder, neoplasm, or carcinoma; but also may be used in-vivo as a preventative procedure performed prophylactically in advance of the patient receiving a clinical diagnosis of breast malignancy (e.g., ductal, medullary, or lobular carcinoma). It can also be used to treat a breast with atypia, premalignancy, carcinoma in-situ, or carcinoma (when not treated with total mastectomy), and can be used as a prophylactic treatment in the contralaterial breast.

### PRIORITY CLAIM

The invention was first filed as U.S. Provisional Patent Application Serial No. 61/124,621 on Apr. 18, 2008. The priority and legal benefits of this first filing are expressly claimed herein.

### FIELD OF THE INVENTION

The present invention is concerned with medical procedures and techniques for treating human breast carcinomas; and is directed particularly to a clinical method using a modified ductal lavage technique and at least one kind of Complement-fixing antibody specific for antigens, haptens, or epitopes which are present on normal, atypical and malignant breast epithelial cells in-vivo.

### BACKGROUND OF THE INVENTION

Initially, it is most important to remember not only what is the relevant anatomy of the human breast (both female and male); but also to understand properly what are the three-dimensional organizational patterns existing within the breast duct anatomy.

### Anatomy of the Human Breast

Anatomically, the human breasts sit on the chest muscles that cover the ribs. Classically each breast has been believed to be composed of 15 to 20 lobes; more recently however, many physicians have proposed that there are actually far fewer numbers of lobes that collectively form the breast.

Typically, each lobe in the breast contains many smaller lobules and a system of discrete ducts; and employs fat (or fat containing cells) to fill in the cavity spaces which exist between the lobules and ducts. The lobules themselves contain groups of tiny glands that produce milk; and the milk released from these glands flows from the lobules and is carried through the ductal system to the nipple. The nipple itself lies in the center of a dark area of skin, the areola.

The number, distribution, and anatomic properties of the ductal systems of the breast, which extend from the nipple orifices to the terminal ducts in each lobule, have recently become better understood in organization and are of increasing medical interest. Reported research studies have shown that more than 90% of all nipples contain about 5-9 ductal orifices, which are generally arranged as a central group and a peripheral group; and each nipple communicates with a separate, non-anastomosing ductal system, which extends to the terminal duct lobular unit [see for example, Cancer 101(9):1947-1957 (Nov. 1, 2004)].

In addition, normal human breast glands and ducts are composed of three cell types that express different subsets of proteins. These cell types are: luminal, basal, and myoepithelial. The luminal and basal cell types express different kids of cytokeratins (CKs); and myoepithelial cells (MECs) express various basal cell-type CKs as well as other more specific markers, such as smooth muscle actin, calponin, and p63.

### The Risk of Breast Cancer

Breast cancer is the most common malignancy among women: In the United States, there are approximately 180,000 cases and 46,000 deaths occurring each year; and worldwide, there are 465,000 breast cancer deaths each year. These breast tumors are comprised of phenotypically diverse populations of breast cancer cells, each capable of becoming its own particular type or kind of cancer.

The lifetime risk of developing breast cancer for women in North America is 1 in 8. Although the number of cases of breast cancer has increased in recent years, mortality rates have actually decreased. This trend toward better overall survivorship is due to increased surveillance, improved diagnostics and better adjuvant therapies.

### Distinguishable Types of Breast Cancer

Breast cancer originates in breast epithelium and is associated with progressive molecular and morphologic changes. Women are at far greater risk than men; and women with atypical breast ductal epithelial cells are recognized as having a markedly increased relative risk of breast cancer.

The vast majority of female breast malignancies are ductal and lobular carcinomas. Both of these carcinoma types are derived from the epithelial cells that line the several individual compound alveolar glands that form tree-like branching tubules and have lactiferous ducts that open onto the surface of the nipple. The remainder and bulk of female breast tissue is constituted of fibrovascular adipose tissue and skin, and is uninvolved with the origination of these carcinomas. Such ductal and lobular carcinomas or malignancies generally occur at an age after breast feeding of children may have occurred.

Ductal carcinoma in situ (“DCIS”) of the female breast was considered to be a rare occurrence prior to the introduction of screening mammography as a diagnostic tool. Today however, DCIS represents about 20-30% of all clinically diagnosed female breast cancers.

Medullary carcinoma of the female breast comprises approximately 2% of invasive breast carcinomas. It is believed to be derived from the myoepithelial cells which lie next to those epithelial cells that line the ducts and lobules of the breast.

In comparison, male breast cancer is an uncommon event, and today represents only about 1% of all breast cancer cases. It has been also noted that, in these instances, many of these male patients have significant family histories of breast cancer, particularly those who show germline mutations of the BRCA1 & BRAC2 genes.

### Diagnostic Test Practices

Today, labeled enzyme conjugated monoclonal antibodies are routinely used in hospital pathology laboratories for immunocytologically diagnosing ductal and lobular carcinomas. The clinically diagnostic immunohistochemical methods use monoclonal or polyclonal antibodies to detect specific antigens or epitopes as markers (or indicators) of clinical tumors present in ex-vivo tissue samples or cell specimens taken from particular anatomic sites in the living patient. Such prepared monoclonal antibodies will specifically attach to specific epitopes existing on the exposed surfaces of ductal and lobular carcinoma cells. The binding of the antibody to specific cells in the patient's tissue sample ex-vivo is then visualized via a chromogenic reaction by linking the antibody-binding reactions to a specific enzyme such as peroxidase. As a consequence of the ex-vivo chromogenic reaction or staining, the location of the targeted cells and antibody-bound protein can then be determined by light microscopy.

Immunohistochemical cytological assays are sensitive, rapid, easy to interpret; and can be performed on frozen or paraffin-embedded tissue taken from the patient. The clinical cytological or histopathological diagnosis is made by a skilled practitioner (typically a trained cytologist or pathologist) who observes and evaluates the morphological structure of the stained cells in the patient's sample under a light microscope. The limitations of such immunocytological assays include the loss of antigenic determinants because of improper fixation or overfixation of tissues, and the subjectivity of interpretation.

When conducting these assays, some of the commonly available enzyme labeled antibodies employed for staining the tissue and cellular components are directed against the following antigens, haptens, or epitopes: a range of different cytokeratins, carcinoembryonic antigen (CEA), epidermal growth factor receptor (EGF), epithelial membrane antigen (EMA), estrogen receptor (ER), progesterone receptor (PR), smooth muscle actin (SMA), muscle specific actin, smooth muscle myosin, calponin, p63 protein, p53 protein, and HER2/neu protein.

### Analysis of the Cells Present in the Ex-Vivo Sample

For routine analytical purposes, the cells in the specimen sample obtained using any of these conventionally known fluid collection procedures are observed, evaluated, and divided into several different cytological categories using specific indicia of cancer or precancerous cells. These cytological categories are typically: (i) benign cells; (ii) atypical cells; (iii) malignant cells; and (iv) insufficient cellular material for diagnosis (or “ICMD”).

For making these determinations, the cells retrieved or collected from the breast duct as ex-vivo test specimens are evaluated for one or more indicia selected from the group consisting of: cell grouping, cell shape, cell size, nuclear size, nuclear shape, presence or absence of nucleoli, nuclear-to-cytoplasmic ratio, vacuoles in the cytoplasm, cytoplasmic shape, cytoplasmic border, presence or absence of anisonucleosis, presence or absence of mitotic figures, nuclear membrane quality, presence of necrotic debris, chromatin distribution, coarseness of chromatin, and the presence or absence of microcalcifications. Based on the presence of any one or more of the observable indicia listed above, the cells in the test sample are then classified as being normal (or benign), or as being atypical (including mild or marked atypia), or as being malignant.

It is noted that atypical cells can typically range in appearance (morphology) from mild atypia to marked atypia. Mild atypia often represents histologic lesions of hyperplasia, atypical ductal hyperplasia, or low-grade ductal carcinomas in-situ (or “DCIS”)—all of which are considered to be lesions of the progressive breast carcinogenesis process. In contrast, the presence of markedly atypical cells indicates the possibility of a neoplastic process, but these cells do not have all the features of malignant cells as yet. Markedly atypical findings can also an indicator of atypical hyperplasia or low-to-intermediate grade DCIS. Atypical cells are also associated with atypical lobular hyperplasia and with lobular carcinoma in situ (or “LCIS”).

### Assessment of Therapeutic Treatment Options

Several factors have been useful in making clinical decisions concerning the available therapeutic treatment options and the risk of disease recurrence in patients diagnosed with early stage breast cancer. These factors include: tumor size, histologic grade, steroid hormone receptor status, DNA ploidy, proliferation rate, cathepsin D expression and expression of particular growth factor receptors.

### Patient age

Among the most important factors associated with an increased risk for the development of human breast cancer is age. A woman's breast cancer risk increases throughout her life. The current incidence of breast cancer is 27.9 cases per 100,000 women per year at 30-35 years of age; whereas the incidence rate increases to 412 cases per 100,000 in women 80 years of age.

### Family History

Another of the important risk factors for breast cancer is the woman's family history. It is known that 5%-10% of breast cancers are due to inherited genetic mutations. Two of these genes, BRCA1 and BRCA2, have been recently cloned and are estimated, together, to be involved in 60%-70% of all hereditary breast cancers [see for example, Banu Arun, “Ductal Lavage and Risk Assessment of Breast Cancer”, The Oncologist, 9(6): 599-605, (November, 2004)]. Women with mutations of these genes have an approximately 50%-80% lifetime risk of developing breast cancer. Germlne mutations in the tumor suppressor gene p53 are also associated with increased breast cancer risk and account for 1% of breast cancers in young women.

However, it is necessary to point out that most women with a family history of breast cancer do not have genetically inherited disease; it is important to distinguish this group from the genetically inherited group, since the former carries a lower lifetime risk for developing breast cancer. In fact, a 30-year-old woman with a mother and sister diagnosed with unilateral breast cancer has up to an 18% lifetime risk of having breast cancer. This risk increases to 25% if her mother and sister had bilateral breast cancer [see for example, Banu Arun, “Ductal Lavage and Risk Assessment of Breast Cancer”, The Oncologist, 9(6): 599-605, (November, 2004)].

### Hormonal Effects

Epidemiologic data also strongly suggest an association between ovarian hormones (estrogen, progesterone, etc.) and the risk of breast cancer. This linkage is supported by the observation that prolonged estrogen exposure—such as with early menarche, late menopause, nulliparity, and late age at first pregnancy—is associated with a higher risk for breast cancer. For example, menarche at age 16 is associated with a 10%-30% reduction in breast cancer risk; and pregnancy at a young age, especially before the age of 20, also reduces the risk for subsequent breast cancer. Studies reporting on the relationship between breast cancer risk and abortion are controversial, as are studies on lactation and breast cancer risk; recent data suggest that prolonged lactation can reduce breast cancer risk [see for example, Banu Arun, “Ductal Lavage and Risk Assessment of Breast Cancer”, The Oncologist, 9(6): 599-605, (November, 2004)].

### Current Clinical Techniques used for Collecting Cells from the Ducts in the Breast

The histological and cytological features of breast epithelial cells and the subsequent risk of breast cancer have been investigated for over 30 years. The finding of precancerous atypical cells or “atypia” can be demonstrated by core biopsy, or by nipple aspiration (“NA”), or by ductal lavage; and the presence of atypical cells in the specimen sample is associated with an increased risk of developing breast carcinoma.

### Nipple Aspiration

Nipple aspirate fluid (“NAF”) can be obtained from non-lactating women by simple suction methods. Several groups have examined the cytology of NAF in attempts to improve detection of breast cancer and predict risk based on cellular findings. Although abnormal cytology in NAF is predictive of subsequent development of breast cancer, with a relative risk of 2.5-5 at 12.5 years, the typical cellular yield provided by this technique is quite low.

Atypia seen in fine-needle aspiration (“FNA”) of the breast—by which multiple aspirations are performed and pooled for analysis—is associated with an increased risk for breast cancer, with a relative risk of 5 at 45 months. The relative risk seen with atypia detected either in fluids obtained by NAF or FNA is consistent with that observed for atypical ductal hyperplasia when detected in surgical specimens; and this reported evidence suggests that atypia (as obtained and identified by these two techniques) may help further define individual risk for breast cancer.

### Ductal Lavage

It has been recognized that the human breast is a self-renewing organ/tissue in which epithelial cells lining the ductal-alveolar mammary tree are exfoliated into the luminal compartments of the gland. Breast ductal lavage is a relatively new approach for collecting and employing these exfoliated cells for early breast cancer detection and risk assessment. In this technique, an individual ductal orifice at the nipple is cannulated with a microcatheterto flush the associated ductal system with saline. The harvested fluids contain thousands of exfoliated epithelial cells that can be evaluated for breast cancer-associated abnormalities.

The ductal lavage technique was clinically developed as a long needed improvement upon the NAF and FNA collection procedures. Even though it is more convenient to obtain NAF and FNA specimens, a major problem and danger of the nipple aspiration approach is that it all too often yields an inadequate number of epithelial cells in the test sample for an accurate and reliable cytopathologic analysis and determination. In meaningful effect therefore, the NAF and FNA collection procedures simply fail to provide a sufficient number of cells in the fluid specimen taken from the patient and used as a test sample, and require either follow-up radiological studies over time or biopsy. For primarily these reasons (although there are others), the diagnostic test results using test specimens obtained by NAF and FNA collection procedures are unfortunately sometimes irregular in outcome, are often not reproducible within acceptable test limits, and are frequently erroneous in accuracy and/or precision.

In comparison, the ductal lavage approach purposely infuses fluid at the nipple, which then enters into the tubular ductal and lobular system of the breast; and such fluid is subsequently withdrawn and collected again as an ex-vivo sample. The ductal lavage technique has been demonstrated to provide ex-vivo fluids which contain far larger numbers and sufficient test quantities of epithelial cells in the collected sample—adequate numbers of cells that can then be examined under the microscope for histological characteristics and be immunologically evaluated to see if any of them are atypical or malignant cells. The ductal lavage collection procedures thus provide adequate numbers of cells for diagnostic testing; and will yield test sample results that are reliable, are reproducible, and are accurate. In the appropriate clinical setting, it will yield useful clinical data.

Ductal lavage as a collection technique depends on the underlying premise that most breast cancers (about 95%) develop in those cells that line the tubular ductal and lobular systems of the breast. Cancer usually begins in one duct and may be contained to that duct if caught early, thereby making a prescribed course of treatment more effective and increasing the rate of patient survival. Many doctors also believe that performing ductal lavage as a screening tool in women at high risk for breast cancer, followed by immunocytological analysis of the obtained cells, may identify breast cancer in its earliest stages—the time when it is most treatable.

Accordingly, ductal lavage is today merely an alternative approach for the collection of increased numbers of cells from the milk ducts of the breast, after which the collected cells are then diagnostically examined and evaluated for specific immunological and cytological features. Clearly, the ductal lavage technique is employed solely for the collection of exfoliated epithelial cells from the ducts of the human breast; and the collected fluid obtained via this approach is utilized only as ex-vivo fluid test samples for the identification and characterization of cancerous and atypical epithelial cells. Equally important, ductal lavage is presently performed for only on those select women patients who have already exhibited multiple breast cancer risk factors, in order to try to detect whether or not definitive breast cancer cells exist at a time well before a palpable tumor can be seen, and before a biopsy target can be identified.

### SUMMARY OF THE INVENTION

The present invention is a method for treating a living human patient believed to be presently or suspected of becoming subsequently afflicted with an identifiable type of atypical or abnormal epithelial cell in the breast, said method comprising the steps of:

identifying at least one duct in the nipple of the human breast as a target for treatment;

introducing a predetermined quantity of a formulated treatment fluid into the lumen of said identified duct, wherein said introduced treatment fluid comprises
(i) at least one Complement-fixing antibody directed against and able to bind to specific antigens, haptens, or epitopes which are characteristically present on normal, atypical, or malignant breast epithelial cells in-vivo,
(ii) Complement proteins forming the chemical components of Complement and sufficient for initiating the cascade of reactions for in-vivo activation and fixation of Complement by said antibody after becoming bound to a breast epithelial cell, and
(iii) a biocompatible fluid carrier;

allowing said introduced treatment fluid to react in-situ for a preselected time period with such breast epithelial cells as are then present within the lumen of said identified duct, whereby at least a portion of said antibodies in said introduced treatment fluid become bound to the breast epithelial cells, and there is an in-situ activation and fixing of Complement proteins by said antibodies after becoming bound to the breast epithelial cells; and

removing said reacted treatment fluid from the lumen of said identified duct in the human breast,

The clinical outcome and desired results produced by the treatment method include:
(a) cell membrane disruption in-vivo of at least some breast epithelial cells then present within said identified duct,
(b) specific cell lysis in-vivo of at least some breast epithelial cells then present within said identified duct,
(c) a denuding of at least some of the normal, atypical and abnormal cells from the epithelium in-vivo then present within said identified duct, and
(d) scarring and normal cell repair for the denuded surface of the epithelium present within said identified duct.

Accordingly, as defined above, the instant invention is an in-vivo treatment method directed against living epithelial cells in the human breast, and uses a modified ductal lavage technique to infuse a purposely formulated treatment fluid into pre-chosen individual ducts in the human breast for reaction with such epithelial cells as then are present within the ducts. The treatment method can be employed as a therapeutic process in-vivo against presently existing breast carcinomas; but alternatively may be used in-vivo as a preventative or prophylactic procedure performed in advance of the patient receiving a clinical diagnosis of breast carcinoma.

### BRIEF DESCRIPTION OF THE FIGURES

The present invention can be more easily understood and more readily appreciated when taken in conjunction with the accompanying Drawing, in which:

FIG. 1 is a pictorial image of the different proteins involved in the fixation of Complement;

FIG. 2 is a chart illustrating the Complement system of more than 30 serum and cellular proteins, including positive and negative regulators, and which are linked in two distinct biochemical cascades of reactions, the classical and alternative pathways; and

FIG. 3 is a schematic representation of the structure of six members of the Regulators of Complement Activation (RCA) family and CD59.

### DETAILED DESCRIPTION OF THE INVENTION

The present invention is a treatment method which uses a modified ductal lavage technique to infuse a purposefully formulated treatment fluid to individual ducts in the human breast for immunological contact and reaction with such living epithelial cells as are then present in the living tissues. The treatment method can be employed as a therapeutic process in-vivo, but may alternatively also be used in-vivo as a preventative procedure performed in advance of receiving a clinical diagnosis of breast carcinoma.

The treatment method of the present invention utilizes at least one preselected Complement-fixing antibody (or preferably an admixture of different Complement-fixing antibodies) and also contains the recognized system components for activating human Complement, both of which are prepared in advance as a free flowing liquid solution or suspension fluid. The minimal components of the prepared treatment fluid will therefore comprise: at least one preselected Complement-fixing antibody; those chemical entities forming the system components of Complement and for initiating the Complement fixation cascade of reactions; and a biocompatible fluid carrier, typically a physiological strength aqueous based liquid.

By infusing the ducts of the breast with the formulated treatment fluid using a modified ductal lavage technique, and then allowing the infused liquid to react in-vivo for a predetermined time with the epithelial cells then present as the cellular lining within the duct and the exfoliated cells then existing within the lumen of the duct; the Complement-fixing antibodies in the treatment fluid will specifically bind with and become attached to the normal cells, the atypical cells, and the abnormal epithelial cells then existing in-vivo within the individual ducts. The protein components of Complement also then present in the liquid treatment fluid will then become activated and be fixed in-situ by the antibodies then specifically bound in-vivo to the normal, atypical and abnormal epithelial cells in the duct.

This manner of in-situ activation and fixing of Complement proteins by the antibodies then bound to duct epithelial cells in-vivo will cause and result in at least one, and preferably all, of the following events: (1) Epithelial cell membrane disruption in-vivo; (2) Specific cell lysis in-vivo; (3) A denuding of the normal, atypical and abnormal cells from the internal epithelium lining of the ducts in-vivo; and (4) Scarring and normal cell repair for the denuded surface lining then present within the ducts. Any and all of these in-vivo events are a direct outcome and consequence of this method of treatment.

### I. The Different Kinds and Pathologies of Breast Disorders and Abnormalities which are Treatable using the Present Methodology

### Δ Breast Neoplasms

A diverse range and variety of different breast neoplasms are deemed to be beneficially treatable, either therapeutically or prophylactically, using the formulated treatment fluids and series of manipulative steps comprising the method of the present invention. These different forms of breast neoplasms may occur either with (being hereditary or familial forms) or without (being sporadic forms) a familial background.

In accordance with the commonly accepted practices used today for identifying and characterizing the various clinical forms of breast neoplasms, the different kinds of neoplasms suitable for treatment include any and all of those listed below:

### A. Benign Epithelial Lesions

Collectively as a class, benign epithelial lesions have no significant tendency to malignant transformation. Among the different categories or types of benign epithelial lesions are the following:

1. Adenomas
ductal
lactating
tubular

2. Adenosis
apocrine
blunt duct
microglandular
sclerosing

3. Fibroadenomas
typical
complex
juvenile

4. Radial scar/complex sclerosing lesions

### B. Invasive Breast Carcinomas

This distinct class is divided into two major categories on the basis of differences in their individual cytoarchitectural features. Note also that the terminology “invasive ductal carcinoma” and/or “lobular carcinoma” does not imply an origin from either ducts or lobules, but indicates instead the presence of cytoarchitectural and phenotypical features of ductal-type and lobular-type, respectively.

1. Invasive ductal carcinomas
acinic cell carcinoma
adenoid cystic carcinoma
apocrine carcinoma
cribriform carcinoma
glycogen-rich/clear cell
inflammatory carcinoma
lipid-rich carcinoma
medullary carcinoma
metaplastic carcinoma
micropapillary carcinoma
mucinous carcinoma
neuroendocrine carcinoma
oncocytic carcinoma
papillary carcinoma
sebaceous carcinoma
secretory breast carcinoma
tubular carcinoma

2. Lobular carcinomas
classic
pleomorphic
trabecular
alveolar
solid

### C. Precursor Lesions of Invasive Breast Carcinoma

The commonly recognized precursor lesions indicative of invasive breast carcinoma are:

1. Intraductal proliferative lesions
atypical ductal hyperplasia
ductal carcinoma in situ
florid ductal hyperplasia
usual ductal hyperplasia

2. Lobular neoplasia/atypical lobular
atypical ductal hyperplasia
   Lastly, the following clinical observations are also well documented:
(i) Ductal adenocarcinoma is the most commonly occurring type of malignant neoplasm.
(ii) Lobular carcinoma is the second most common malignant breast tumor.
(iii) Medullary carcinoma is a rarer occurrence.
(iv) Hyperplasia as such is merely a proliferation of cells without criteria of malignancy. Note that hyperplasia is any proliferation of ductal or lobular epithelial cells, without criteria of malignancy; in contrast to atypical hyperplasia, which has incomplete malignant features and can be difficult to distinguish from in-situ carcinoma.
(v) Fibroadenomas are benign breast tumors. Note also that Fibroadenomas are the most common form of benign breast tumors.

### Δ Pathologies

### α. Invasive Breast Carcinomas

The most common histologic types of invasive breast carcinoma are designated as ductal carcinoma or “not otherwise specified” (NOS) carcinoma. These instances comprise about 80% of all clinical cases.

Ductal or NOS tumors typically are a proliferation of epithelial cells from galactophoral ducts; it may be preceded and accompanied by an in-situ component characterized by a proliferation of cells within the ducts without interruption of the basal membrane; and when this membrane is altered, the carcinoma is invasive. These histological types can be graded on the basis of the architecture (amount of tubule formations), nuclear atypia and mitotic activity. The score resulting from the sum of these criteria (i.e., Nottingham score) has been show to be an important prognostic indicator.

A 3-grade system has been developed for in-situ ductal carcinoma. Grade III in situ ductal carcinoma corresponds to the classic comedocarcinoma; whereas Grade I in-situ ductal carcinoma merges with atypical ductal hyperplasia, to the point that some physicians regard the two processes as being one and the same. Recently, a proposal has been made to embrace all of the various types of intraductal proliferative lesions under the term ductal intraepithelial neoplasia (DIN), and to divide this category into 3 different grades, each having an increasingly higher risk for the development of invasive carcinoma.

Many morphologic variants (or subtypes) of invasive ductal carcinoma are known to exist, some of them being extremely rare. The recognized listing of subtypes currently includes the following carcinomas: Tubular, cribriform, medullary, mucinous, neuroendocrine, papillary, micropapillary, apocrine, metaplastic, lipid-rich, secretory, oncocytic, adenoid cystic, acinic cell, glycogen-rich (clear cell), sebaceous, and inflammatory carcinoma. The prognosis of these subtypes varies, some of them having a better and some a worse outcome than invasive ductal carcinoma, NOS.

### β. Invasive Lobular Carcinomas

Invasive lobular carcinoma is the second major and most commonly occurring type (5-10%) of breast cancer. Like its ductal counterpart, invasive lobular carcinoma may be preceded or accompanied by an in-situ component. In addition, invasive lobular carcinoma is histologically more homogeneous than ductal carcinoma; but some morphologic variation exist, such as the pleomorphic and classic variants.

### γ. Medullary Carcinomas

Medullary carcinoma is not only a relatively rare form of ductal carcinoma (2%), but has a much better prognosis. Medullary carcinoma has a very distinctive morphology: It is sharply circumscribed; is often accompanied by a heavy lymphoid infiltrate of high nuclear grade with a syncytial pattern of growth; and lacks in-situ or microglandular features. When one or more of these distinctive features is lacking, the tumor is referred to as Òatypical medullary carcinoma”. Note also that a high frequency of medullary carcinoma has been reported in patients afflicted with BRCA1 germ line mutations.

### II. The Minimal Steps Comprising the In-Vivo Treatment Method as a Whole

The therapeutic or prophylactic treatment method is a minimally invasive in-vivo procedure that may be performed in a doctor's office or outpatient center. The treatment method is a substantive modification of that procedure now commonly employed by physicians for ductal lavage; and the innovative treatment method is minimally performed as a series of four manipulative steps:

### Step 1: Identification of the Individual Ducts to be Treated.

For the comfort of the patient, an anesthetic cream is first applied to numb the nipple area of the breast. Gentle suction using conventional equipment (such as a syringe or microcatheter) is used to withdraw a small amount of fluid from the milk ducts. This act is performed to locate the opening of the individual ducts as they exist on the nipple surface, and to identify which and how many individual ducts among the number actually present are to be used for treatment purposes.

Step 2: Introduction of a Prepared Antibody Treatment Fluid as a In-Vivo Reactant into the Lumen of each Individual Duct.

Preferably, a hair-thin catheter (typically a microcatheter having a very small outer and inner lumen diameter) is carefully inserted into the natural opening of each identified duct. Additional anesthetic (such as 1% lidocaine) is also delivered into each individual duct used as a target in the procedure, for the added comfort of the patient.

Then, a formulated therapeutic or preventative treatment fluid (comprising at least one Complement-fixing antibody and sufficient Complement proteins) is then infused (or otherwise introduced) through the catheter into the lumen of the individual ducts. The introduced treatment fluid then washes over and immunologically reacts with the living epithelial cells then present within each individual duct—an in-vivo act which not only causes direct interaction and reaction with the exfoliated cells then present within the lumen of the ducts, but also initiates direct antibody binding with and adherence to those epithelial cells then forming the live cellular lining disposed upon the internal surface of the duct wall.

Step 3: Allowing the Introduced Treatment Fluid to React for a Predetermined Time within the Individual Duct.

The formulated therapeutic or preventative treatment fluid is then allowed to react with the all the epithelial cells then present within the individual duct. These breast epithelial cells include the exfoliated cells then present within the lumen of the ducts, as well as those breast epithelial cells then forming the live cellular lining disposed upon the duct wall.

It is also intended and expected that the introduced fluid will be agitated (via light suction and/or direct physical manipulation of the breast) such that the epithelial cells lining the individual duct are repeatedly washed by, reacted with, and become bound at least in part by the antibodies present within the introduced treatment fluid.

Step 4: Removal of the Treatment Fluid from the Duct after a Pre-Chosen Time has Passed for In-Vivo Reactions to Occur.

After a predetermined period of time for reactive immunological contact and reaction has elapsed, the treatment fluid is slowly withdrawn from the lumen of each individual duct—usually by applying gentle vacuum suction to the catheter.

It will be noted and appreciated also that the final step of removing the treatment fluid (after immunological contact and reaction has occurred) achieves not less than four (4) very desirable outcomes and consequences for the methodology as a whole. These are:

(a) The in-vivo immunological reactions with breast epithelial cells are halted because the Complement-fixing antibodies and the Complement proteins have been physically removed from the lumen of each individual duct;

(b) The exfoliated epithelial cells and such epithelial cells as have become released from the cellular lining of the ducts during the course of performing the treatment process are concomitantly removed by this removal action;

(c) Many of the epithelial cells then retained as part of the cellular lining for the individual ducts have been internally damaged, or have become disrupted (fragmented in part or in whole), or have been lysed (completely ruptured or disintegrated) in-situ as a direct consequence of the treatment; and

(d) A healing and scarring process is begun in-vivo which will replace the epithelial cells of the ducts and lobules.

### III. The Minimal Constituents of the Formulated Treatment Fluid

The minimal components of the prepared in advance treatment fluid will include and comprise: Those chemical compounds and compositions forming the reactive protein system and cascade of sequential reactions for Complement fixation; at least one Complement-fixing antibody specific against a breast epithelial cell antigen, hapten, or epitope; and a biocompatible fluid carrier.

### A. Complement Proteins & Complement Activation

Complement is a term originally used to refer to the heat labile factor in serum that causes immune cytolysis—i.e., the lysis of antibody coated cells; and now commonly refers to the entire functionally related system comprising many distinct serum proteins that collectively serve as the effector not only of immune cytolysis, but also of other biologic functions.

Complement activation can be initiated and occur by either of two different pathways and reaction sequences: the classic pathway, and the alternative pathway.

### The Complement Protein System, its Constituents, and its Regulation

The Complement system consists of a series of about 25-30 proteins that act in combination with antibodies to protect cells and tissues, and help rid the body of antigen-antibody complexes formed in-vivo.

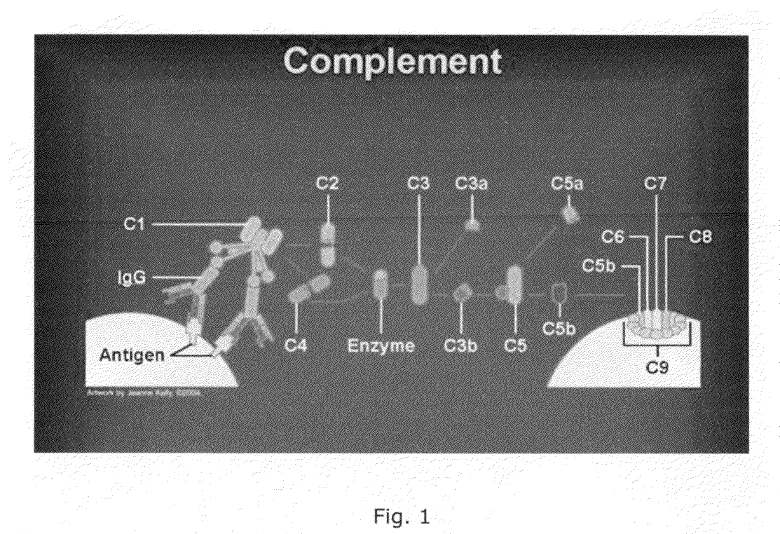

Under in-vivo circumstances, Complement proteins circulate within the blood of a mammal in an inactive form. The so-called “Complement cascade” or series of sequential reactions begins when the first complement molecule, termed “C1”, encounters and interacts with antibody then bound to an antigen on the surface of a target cell in an antigen-antibody complex. Each of the other Complement proteins, identified as C2 through C9 respectively, then performs its part in reaction sequence, with each product then acting in turn on the molecule next in the reaction sequence. The end result is a disruption and rupture of the cell membrane, which allows fluids and other molecules to flow in and out of the target cell. The Complement cascade of reactive entities and sequential reactions is illustrated by FIG. 1.

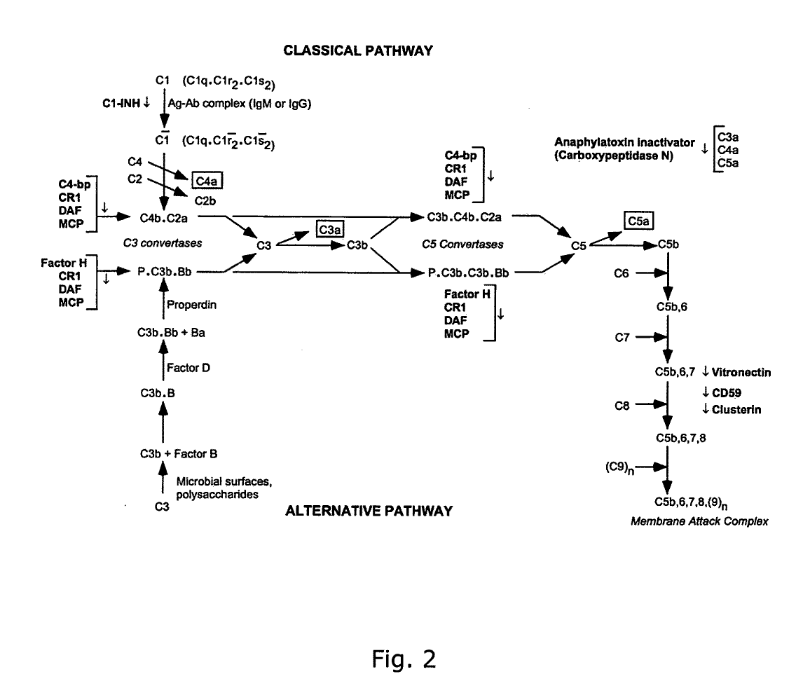

The Complement system typically includes 25-30 serum and cellular proteins; including both positive and negative regulators, linked in two biochemical cascades, termed the “classical” and “alternative” pathways. The dual systems of classical and alternative pathways and their individual series of sequential interdependent protein reactions are illustrated by FIG. 2.

As shown by FIGS. 1 and 2 respectively, the activation and fixation of Complement encompasses a series of initiation, amplification, and lytic steps as discrete reactions (Parker, 1992; Liszewski et al., 1996). The cascade system of reactions is regulated at multiple levels temporally as well as spatially. This regulation facilitates recognition of self from foreign tissue (Farries and Atkinson, 1987) and, therefore, allows for control over the potent tissue-damaging capabilities of Complement activation.

The classical pathway is normally activated by complexes of one or more antigens existing on the surface of a target cell and IgM or IgG antibody classes. The alternative pathway is activated by microbial surfaces and complex polysaccharides—e.g., yeast cell walls, endotoxins, viral particles.

As FIG. 2 shows, in both the classical and alternative pathways, C3 is converted into C3b by the C3 convertases; whereas in the classical pathway C5 is converted into C5b by the C5 convertases. The three anaphylatoxins, C3a, C4a and C5a are released during the various enzymatic reactions of the cascade. The membrane attack complex is formed by the sequential binding of C5b to C6, C7, C8 and C9.

Both pathways are subject to fine regulation by soluble (C1 inhibitor, C4bp, factor H, vitronectin, clusterin) as well as membrane-bound (CR1, DAF, MCP, CD59) proteins. The anaphylatoxins C3a, C4a and C5a are themselves inactivated by carboxypeptidase N.

### Details of the Classical Pathway

In the classic pathway, the reactive proteins are termed “components of Complement”, and are commonly designated by the symbols C1 through C9. Note that C1 itself is a calcium dependent complex of three distinct proteins—C1q, C1r, and C1s respectively. The classic pathway is activated by the binding of C1 to classic pathway activators, primarily antigen-antibody complexes containing IgM, IgG1, IgG2, IgG3, and IgG4. C1q binds to a single IgM molecule or two adjacent IgG molecules [see for example, Savvas C. Makrides, “Therapeutic Inhibition of the Complement System”, Pharmacological Reviews, 50(1): 59-88 (March 1998)].

### Summary of the Classical Pathway

FIG. 1 illustrates the details of the classical Complement system and its regulatory proteins. As shown by FIG. 1, the classical pathway is activated by complexes of antigen and IgM or IgG antibody classes. In comparison, the alternative pathway is activated by microbial surfaces and complex polysaccharides, e.g., yeast cell walls, endotoxins, viral particles.

In the classical pathway, C3 is converted into C3b by the C3 convertases; and C5 converted into C5b by the C5 convertases. The three anaphylatoxins, C3a, C4a and C5a are released during the various enzymatic reactions of the cascade. The membrane attack complex is formed by the sequential binding of C5b to C6, C7, C8 and C9.

The classical pathway as a series of sequential reactions is subject to fine regulation by soluble proteins such as C1 inhibitor, C4bp, factor H, vitronectin, clusterin; as well as membrane-bound proteins such as CR1, DAF, MCP, CD59. Also, the anaphylatoxins themselves are inactivated by carboxypeptidase N.

For general benefit and use, a more detailed listing of the regulatory proteins for the Complement pathway is given by Table 1 below.

### Reaction Sequences

The classical pathway illustrated by FIG. 1 is usually initiated when a complex of antigen and IgM or IgG antibody binds to the first component of Complement C1. Activation of this step of Complement is regulated by the C1 inhibitor, which binds to C1 r and C1s and dissociates them from C1q (Liszewski et al., 1996). Activated C1 cleaves both C4 and C2 to generate C4a and C4b, as well as C2a and C2b. The C4b and C2a fragments combine to form the C3 convertase, which, in turn, cleaves the third component of Complement, C3, to form C3a and C3b. The binding of C3b to the C3 convertase yields the C5 convertase, which cleaves C5 into C5a and C5b, the latter becoming part of the membrane attack complex (MAC).

The three peptides released during these particular reactions—i.e., C3a, C4a, and C5a respectively—are known as anaphylatoxins (Hugli and Muller-Eberhard, 1978); and they differ in their relative potencies. C5a is the most potent anaphylatoxin; followed by C3a, which, in turn, is 10- to 100-fold more active than C4a (Cui et al., 1994; Hugli and Muller-Eberhard, 1978; Liszewski et al., 1996).

The anaphylatoxins mediate multiple reactions in the acute inflammatory response, including smooth muscle contraction,.changes in vascular permeability, histamine release from mast cells, neutrophil chemotaxis, platelet activation and aggregation (Morgan, 1986; Hugli, 1989; Gerard and Gerard, 1994), as well as up-regulation of adhesion molecules that can also play key roles in neutrophil recruitment (Foreman et al., 1994; Mulligan et al., 1996, 1997; Schmid et al., 1997a).

Recently, C3a and C5a have been shown to be potent chemotactic factors for human mast cells (Hartmann et al., 1997). The anaphylatoxins are rapidly inactivated by carboxypeptidase N, which cleaves the carboxyl terminal arginyl residue from each anaphylatoxin, thus converting them into their des-Arg forms (Bokisch et al., 1969; Bokisch and Muller-Eberhard, 1970; Chenoweth, 1986). A C5a-inactivating enzyme isolated from human peritoneal fluid has been described (Ayesh et al., 1995).

The C3 and C5 convertases of the classical pathway [see FIG. 2] are controlled by members of the Regulators of Complement Activation (RCA) family, which are identified and listed by Table 2 below, This RCA family includes: the membrane-bound regulators Complement receptor type 1 proteins (CR1; C3b/C4b receptor; CD35), Complement receptor type 2 proteins (CR2; CD21; Epstein-Barr virus receptor); membrane cofactor proteins (MCP; CD46; measles virus receptor); decay-accelerating factor (DAF; CD55); and the serum proteins factor H and C4b-binding protein (C4bp).

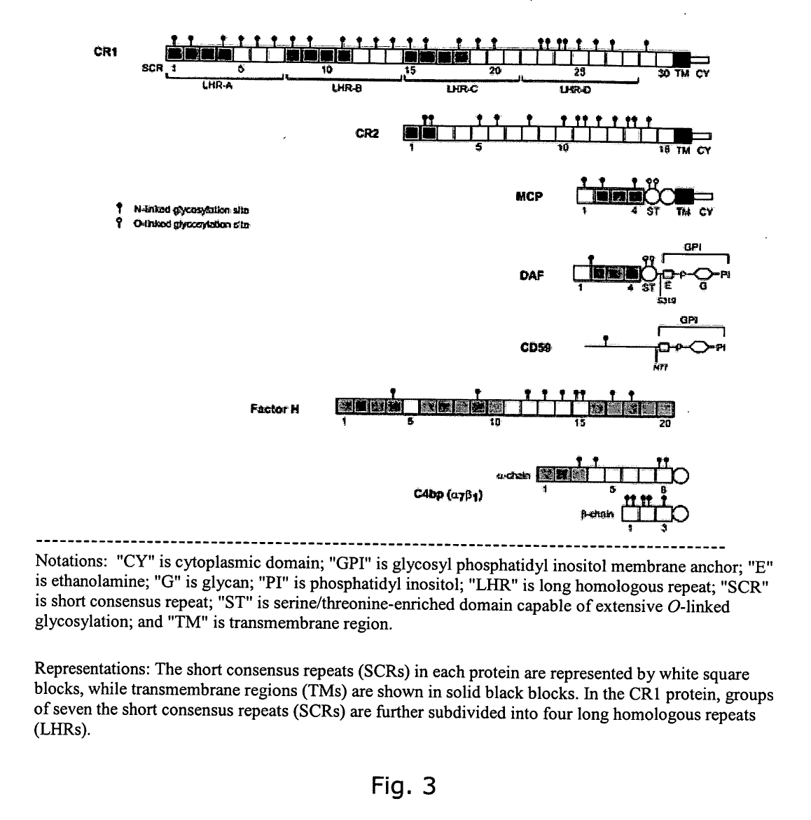

A schematic representation of the structure for six members of the RCA family and CD59 is shown by FIG. 3 herein; note however, that only the common isoforms of these members are illustrated in FIG. 3. As seen therein, the short consensus repeats (SCRs) in each protein are represented by white square blocks, while transmembrane regions (TMs) are shown in solid black blocks. In the CR1 protein, groups of seven short consensus repeats (SCRs) are further subdivided into four long homologous repeats (LHRs).

The number of N-linked glycosylation sites in each protein is also shown by FIG. 3. The indicated N-linked glycosylation sites are based on the cDNA sequence of: CR1 (Klickstein et al., 1988), CR2 (Moore et al., 1987; Weis et al., 1988), MCP (Liszewski et al., 1991), DAF (Caras et al., 1987; Medof et al., 1987), CD59 (Sugita et al., 1989; Davies et al., 1989), factor H (Ripoche et al., 1988), C4bp -chain (Chung et al., 1985), and C4bp -chain (Hillarp and Dahlback, 1990).

The ligand-binding active sites in each protein shown by FIG. 3 are also stippled in the appropriate SCRs for CR1 (Klickstein et al., 1988; Kalli et al., 1991; Makrides et al., 1992); for CR2 (Fearon and Carter, 1995), MCP (Adams et al., 1991); for DAF (Coyne et al., 1992; Kuttner-Kondo et al., 1996); for factor H (Gordon et al., 1995; Sharma and Pangburn, 1996); and for C4bp (Ogata et al., 1993; Hardig et al., 1997).

Note also that with factor H, only the first C3b-binding site (SCRs 1-4) exhibits factor I cofactor activity (Sharma and Pangburn, 1996). Factor H also contains two heparin-binding sites, one near SCR 13 and another in SCR 6-10 (Sharma and Pangburn, 1996) or SCR 7 (Blackmore et al., 1996).

Lastly, the amino acid residues in the active site of CD59 have also been identified by FIG. 3 (Zhou et al., 1996; Yu et al., 1997).

TABLE 1
Regulatory proteins of the Complement pathway

| Protein | Location | Ligand | Function/activity | References |
|---|---|---|---|---|
| C1-inhibitor | Plasma | C1r, C1s | Dissociates C1; regulates the | Davis, 1988; Davis et al., |
| | | | contact (kinin-forming | 1993 |
| | | | pathway); a serpin (Serine | |
| | | | Protease Inhibitor) | |
| Factor I | Plasma | C4b, | Cleaves and inactivates C4b and | Goldberger et al., 1987; |
| | | C3b | C3b using CR1, MCP, C4bp, or | Catterall et al., 1987; |
| | | | factor H as cofactors | Vyse et al., 1996 |
| Factor H | Plasma | C3b | Accelerates decay of C3 | Whaley and Ruddy, 1976; |
| | | | convertases in alternative | Weiler et al., 1976; |
| | | | pathway; dissociates B and Bb | Ripoche et al., 1988 |
| | | | from C3b; cofactor for cleavage | |
| | | | of C3b by factor I | |
| C4bp | Plasma | C4b | Accelerates decay of C3 | Chung et al., 1985; Gigli |
| | | (C3b) | convertases in classical | et al., 1979 |
| | | | pathway; dissociates C2 and | |
| | | | C2a from C4b; cofactor for C4b | |
| | | | cleavage by factor I | |
| CR1 (CD35) | Membrane | C4b, | Accelerates decay of C3 and C5 | Ahearn and Fearon, 1989; |
| | | C3b, | convertases in classical and | Klickstein et al., 1988; |
| | | iC3b | alternative pathways; dissociates | Krych et al., 1991 |
| | | | C2 and C2a from C4b; | |
| | | | dissociates B and Bb from C3b; | |
| | | | cofactor for cleavage of C4b and | |
| | | | C3b by factor I | |
| CR2 (CD21) | Membrane | iC3b, | B cell receptor for complexes | Ahearn and Fearon, 1989; |
| | | C3dg, | having bound C3 fragments; | Fearon and Carter, 1995 |
| | | C3d | cofactor for cleavage of iC3b by | |
| | | | factor I; Epstein-Barr virus | |
| | | | receptor | |
| MCP (CD46) | Membrane | C3b | Blocks formation of C3 | Seya et al., 1986; Cho et |
| | | (C4b) | convertases in classical and | al., 1991; Naniche et al., |
| | | | alternative pathways; cofactor | 1993; Dorig et al., 1993; |
| | | | for cleavage of C3b and C4b by | Okada et al., 1995 |
| | | | factor I; receptor for measles | |
| | | | virus and Streptococcus | |
| | | | pyogenes | |
| DAF (CD55) | Membrane | C4b, | Accelerates decay of C3 | Fujita et al., 1987; |
| | | C3bA | convertases in classical and | Nicholson-Weller et al., |
| | | | alternative pathways; dissociates | 1982; Nicholson-Weller |
| | | | C2 and C2a from C4b; | and Wang, 1994; Kuttner- |
| | | | dissociates B and Bb from C3b | Kondo et al., 1996 |
| CD59 | Membrane | C7, C8 | Blocks formation of MAC on | Davies et al., 1989; Sugita |
| | | | host cells | et al., 1993; Rollins et al., |
| | | | | 1991 |
| Vitronectin (S- | Plasma | C5b-7 | Blocks formation of fluid-phase | Podack and Muller- |
| protein) | | | MAC | Eberhard, 1979; Jenne |
| | | | | and Stanley, 1985; Suzuki |
| | | | | et al., 1985; Hayman et |
| | | | | al., 1983; Tschopp et al., |
| | | | | 1988; Johnson et al., |
| | | | | 1994; Preissner, 1991; |
| | | | | Sheehan et al., 1995 |
| Clusterin (SP- | Plasma | C5b-9 | Blocks formation of fluid-phase | Kirszbaum et al., 1989; |
| 40,40) | | | MAC | Tschopp et al., 1993; |
| | | | | Rosenberg and Silkensen, |
| | | | | 1995; McDonald and |
| | | | | Nelsestuen, 1997 |
| Anaphylatoxin | Plasma | C5a, | Cleaves terminal arginine | Chenoweth, 1986 |
| inhibitor | | C4a, | residue and inactivates | |
| | | C3a | anaphylatoxins; | |
| | | | carboxypeptidase N | |
| Properdin | Plasma | C3bBb | Binds to and stabilizes C3 | Farries et al., 1988; |
| | | | convertase in alternative | Fearon and Austen, 1975 |
| | | | pathway | |
| Nephritic | Plasma | C3bBb, | Bind to and stabilize the C3 | Spitzer et al., 1969; Daha |
| factors | | C4bC2a | convertases in classical and | et al., 1977; Hiramatsu |
| | | | alternative pathways resulting in | and Tsokos, 1988 |
| | | | chronic C3 cleavage | |

*Reproduced from Savvas C. Makrides, “Therapeutic Inhibition of the Complement System”, Pharmacological Reviews, 50(1): 59-88 (March 1998).

TABLE 2
Functions of proteins of the RCA family

| | | | Factor I | | |
|---|---|---|---|---|---|
| | Decay-accelerating | | cofactor | | |
| | activity | | activity | | |
| Protein | Classical | Alternative | C4b | C3b | Substrate |
| CR1 | + | + | + | + | C3b/C4b |
| CR2 | − | − | − | −^{a} | iC3b/C3dg |
| DAF | + | + | − | − | C3b/C4b |
| MCP | − | − | + | + | C3b/C4b |
| C4-bp | + | − | + | − | C4b |
| Factor H | − | + | − | + | C3b |

^{a}CR2 possesses factor I cofactor activity for iC3b.
*Reproduced from Savvas C. Makrides, “Therapeutic Inhibition of the Complement System”, Pharmacological Reviews, 50(1): 59-88 (March 1998).

### Details of the Alternative Pathway

The proteins of the alternative pathway (also referred to as the “properdin” system) and its Complement regulatory proteins are commonly known by semisystematic or trivial names. Fragments resulting from proteolytic cleavage of Complement proteins are designated with lower case letter suffixes, for example, C3a. Inactivated fragments may be designated with the suffix “I”, for example C3bi. Activated components or complexes with biological activity are designated by a bar over the symbol, as for example C1, or C4b, 2a [see for example, Savvas C. Makrides, “Therapeutic Inhibition of the Complement System”, Pharmacological Reviews, 50(1): 59-88 (March 1998)].

The alternative pathway can be activated by IgA immune complexes and also by non-immunologic materials including bacterial endotoxins, microbial polysaccharides and cell walls. Activation of the classic pathway triggers an enzymatic cascade involving C1, C4, C2 and C3, activation of the alternative pathway triggers a cascade involving C3 and factors B, D and P. Both result in the cleavage of C5 and the formation of the membrane attack complex. This complex achieves cell lysis by cell membrane disruption.

### Summary of the Alternative Pathway

This arm of the Complement system is triggered by microbial surfaces and a variety of complex polysaccharides. C3b, formed by the spontaneous low-level cleavage of C3, can bind to nucleophilic targets on cell surfaces and form a complex with factor B that is subsequently cleaved by factor D (see FIG. 2).

The resulting C3 convertase is stabilized by the binding of properdin (P) that increases the half-life of this convertase (Fearon and Austen, 1975). Cleavage of C3 and binding of an additional C3b to the C3 convertase give rise to the C5 convertase of the alternative pathway (see FIG. 2).

Subsequent reactions are common to both pathways and lead to the formation of the Membrane Attack Complex (MAC). The C3 and C5 convertases of the alternative pathway are controlled by CR1, DAF, MCP, and by factor H. These regulators differ in their mode of action—i.e., their decay-accelerating activity (to dissociate convertases) and their ability to serve as required cofactors in the degradation of C3b or C4b by factor I. Tables 1 and 2 respectively, as given above, provide details of these regulatory proteins. In addition, CR2 is said to have a minor role in regulating Complement activation (Fearon and Carter, 1995).

### The Membrane Attack Complex:

The C5 convertases in both the classical and alternative pathways cleave C5 to produce C5a and C5b. Thereafter, C5b sequentially binds to C6, C7, and C8 to form C5b-8 that catalyzes the polymerization of C9 to form the MAC (Tschopp et al., 1982). This structure inserts into target membranes and causes cell lysis (Hu et al., 1981; Podack et al., 1982). However, deposition of small amounts of the MAC on cell membranes of nucleated cells may mediate a range of cellular processes without causing cell death (Morgan, 1992; Nicholson-Weller and Halperin, 1993; Benzaquen et al., 1994).

Three different molecules are known to be involved in the control of the MAC formation. Vitronectin controls fluid-phase MAC by binding to the C5b-7 complex, preventing its insertion into membranes (Podack et al., 1977). Similarly, clusterin (SP-40,40; cytolysis inhibitor; sulfated glycoprotein 2; apolipoprotein J) (Liszewski et al., 1996) blocks fluid-phase MAC by binding to the C5b-7 complex (Jenne and Tschopp, 1989; Choi et al., 1989; Murphy et al., 1989). CD59 blocks MAC formation by binding to C8 and C9, and inhibiting the incorporation and subsequent polymerization of C9 (Rollins et al., 1991).

An additional protein, homologous restriction factor (Zalman, 1992), may be involved in MAC regulation, but it has been suggested that the functional activity reported for homologous restriction factor might possibly be due to a contamination by CD59 aggregates during purification (Liszewski et al., 1996).

### Cited Scientific Publications

For overall knowledge and general benefit, a complete citation identification and listing of the scientific publications identified above, and particularly by Tables 1 and 2 respectively, is presented below. All of these scientific publications, however, are deemed to be of merely background interest and limited informational value. The scientific publications cited above are:

Adams E M, Brown M C, Nunge M, Krych M and Atkinson J P (1991), “Contribution of the repeating domains of membrane cofactor protein (CD46) of the complement system to ligand binding and cofactor activity”, J Immunol 147: 3005-3011;

Ahearn J M and Fearon D T (1989), “Structure and function of the complement receptors, CR1 (CD35), and CR2 (CD21)”, Adv Immunol 46: 183-219;

Blackmore T K, Sadlon T A, Ward H M, Lublin D M and Gordon D L (1996), “Identification of a heparin binding domain in the seventh short consensus repeat of complement factor H”, J Immunol 157: 5422-5427;

Bokisch V A and Müller-Eberhard H J (1970), “Anaphylatoxin inactivator of human plasma: Its isolation and characterization as a carboxypeptidase”, J Clin Invest 49: 2427-2436;

Bokisch V A, Muller-Eberhard H J and Cochrane C G (1969), “Isolation of a fragment (C3a) of the third component of human complement containing anaphylatoxin and chemotactic activity and description of an anaphylatoxin inactivator of human serum”, J Exp Med 129: 1109-1130;

Caras I W, Davitz M A, Rhee L, Weddell G, Martin D W, Jr. and Nussenzweig V (1987), “Cloning of decay-accelerating factor suggests novel use of splicing to generate two proteins”, Nature 325: 545-549;

Catterall C F, Lyons A, Sim R B, Day A J and Harris T J (1987), “Characterization of primary amino acid sequence of human complement control protein factor I from an analysis of cDNA clones”, Biochem J 242: 849-856;

Chenoweth D E (1986), “Complement mediators of inflammation”, in Immunobiology (Ross G ed) pp 63-86, Academic Press, New York;

Cho S W, Oglesby T J, Hsi B L, Adams E M and Atkinson J P (1991), “Characterization of three monoclonal antibodies to membrane co-factor protein (MCP) of the complement system and quantification of MCP by radioassay”, Clin Exp Immunol 83: 257-261;

Chung L P, Bentley D R and Reid K B (1985), “Molecular cloning and characterization of the cDNA coding for C4b-binding protein, a regulatory protein of the classical pathway of the human complement system”, Biochem J 230: 133-141;

Coyne K E, Hall S E, Thompson E S, Arce M A, Kinoshita T, Fujita T, Anstee D J, Rosse W and Lublin D M (1992), “Mapping of epitopes, glycosylation sites and complement regulatory domains in human decay accelerating factor”, J Immunol 149: 2906-2913;

Cui L, Carney D F and Hugli T E (1994), “Primary structure and functional characterization of rat C5a: An anaphylatoxin with unusually high potency”, Protein Sci 3: 1169-1177;

Daha M R, Austen K F and Fearon D T (1977), “The incorporation of C3 nephritic factor (C3NeF) into a stabilized C3 convertase, C3bBb(C3NeF), and its release after decay of convertase function”, J Immunol 119: 812-817;

Davies A, Simmons D L, Hale G, Harrison R A, Tighe H, Lachmann P J and Waldmann H (1989), “CD59, an LY-6-like protein expressed in human lymphoid cells, regulates the action of the complement membrane attack complex on homologous cells”, J Exp Med 170: 637-654;

Davis A E III (1988), “C1 inhibitor and hereditary angioneurotic edema”, Annu Rev Immunol 6: 595-628;

Davis A E III, Aulak K S, Zahedi K, Bissler J J and Harrison R A (1993), “C1 inhibitor”, Methods Enzymol 223: 97-120;

Dorig R E, Marcil A, Chopra A and Richardson C D (1993), “The human CD46 molecule is a receptor for measles virus (Edmonston strain)”, Cell 75: 295-305;

Farries T C and Atkinson J P (1987), “Separation of self from non-self in the complement system”, Immunol Today 8: 212-215;

Farries T C, Lachmann P J and Harrison R A (1988), “Analysis of the interaction between properdin and factor B, components of the alternative-pathway C3 convertase of complement”, Biochem J 253: 667-675;

Fearon D T and Austen K F (1975), “Properdin: Binding to C3b and stabilization of the C3b-dependent C3 convertase”, J Exp Med 142: 856-863;

Fearon D T and Carter R H (1995), “The CD19/CR2/TAPA-1 complex of B lymphocytes: Linking natural to acquired immunity”, Annu Rev Immunol 13: 127-149;

Foreman K E, Vaporciyan A A, Bonish B K, Jones M L, Johnson K J, Glovsky M M, Eddy S M and Ward P A (1994), “C5a-induced expression of P-selectin in endothelial cells”, J Clin Invest 94: 1147-1155;

Fujita T, Inoue T, Ogawa K, Iida K and Tamura N (1987), “The mechanism of action of decay-accelerating factor (DAF): DAF inhibits the assembly of C3 convertases by dissociating C2a and Bb”, J Exp Med 166: 1221-1228;

Gerard C and Gerard N P (1994), “C5a anaphylatoxin and its seven transmembrane-segment receptor”, Annu Rev Immunol 12: 775-808;

Gigli I, Fujita T and Nussenzweig V (1979), “Modulation of the classical pathway C3 convertase by plasma proteins C4 binding protein and C3b inactivator”, Proc Natl Acad Sci USA 76: 6596-6600;

Goldberger G, Bruns G A, Rits M, Edge M D and Kwiatkowski D J (1987), “Human complement factor I: Analysis of cDNA-derived primary structure and assignment of its gene to chromosome 4”, J Biol Chem 262: 10065-10071;

Gordon D L, Kaufman R M, Blackmore T K, Kwong J and Lublin D M (1995), “Identification of complement regulatory domains in human factor H”, J Immunol 155: 348-356;

Härdig Y, Hillarp A and Dahlbäck B (1997), “The amino-terminal module of the C4b-binding protein -chain is crucial for C4b binding and factor I-cofactor function”, Biochem J 323: 469-475;

Hartmann K, Henz B M, Krüger-Krasagakes S, Köhl J, Burger R, Guhl S, Haase I, Lippert U and Zuberbier T (1997), “C3a and C5a stimulate chemotaxis of human mast cells”, Blood 89: 2863-2870;

Hayman E G, Pierschbacher M D, Ohgren Y and Ruoslahti E (1983), “Serum spreading factor (vitronectin) is present at the cell surface and in tissues”, Proc Natl Acad Sci USA 80: 4003-4007;

Hillarp A and Dahlback B (1990), “Cloning of cDNA coding for the beta chain of human complement component C4b-binding protein: Sequence homology with the alpha chain”, Proc Natl Acad Sci USA 87: 1183-1187;

Hiramatsu M and Tsokos G C (1988), “Epstein-Barr virus transformed B cell lines derived from patients with systemic lupus erythematosus produce a nephritic factor of the classical complement pathway”, Clin Immunol Immunopathol 46: 91-99;

Hugli T E (1989), “Structure and function of C3a anaphylatoxin”, Curr Top Microbiol Immunol 153: 181-208;

Hugli T E and Müller-Eberhard H J (1978), “Anaphylatoxins: C3a and C5a”, Adv Immunol 26: 1-53;

Jenne D and Stanley K K (1985), “Molecular cloning of S-protein: A link between complement, coagulation and cell-substrate adhesion”, EMBO J 4: 3153-3157;

Kalli K R, Hsu P H, Bartow T J, Ahearn J M, Matsumoto A K, Klickstein L B and Fearon D T (1991), “Mapping of the C3b-binding site of CR1 and construction of a (CR1)₂-F(ab′)₂ chimeric complement inhibitor”, J Exp Med 174: 1451-1460;

Klickstein L B, Bartow T J, Miletic V, Rabson L D, Smith J A and Fearon D T (1988), “Identification of distinct C3b and C4b recognition sites in the human C3b/C4b receptor (CR1, CD35) by deletion mutagenesis”, J Exp Med 168: 1699-1717;

Krych M, Hourcade D and Atkinson J P (1991), “Sites within the complement C3b/C4b receptor important for the specificity of ligand binding”, Proc Natl Acad Sci USA 88: 4353-4357;

Kuttner-Kondo L, Medof M E, Brodbeck W and Shoham M (1996), “Molecular modeling and mechanism of action of human decay-accelerating factor”, Protein Eng 9: 1143-1149;

Liszewski M K and Atkinson J P (1996), “Membrane cofactor protein (MCP; CD46): Isoforms differ in protection against the classical pathway of complement”, J Immunol 156: 4415-4421;

Liszewski M K, Post T W and Atkinson J P (1991), “Membrane cofactor protein (MCP or CD46): Newest member of the regulators of complement activation gene cluster”, Annu Rev Immunol 9: 431-455;

Makrides S C, Scesney S M, Ford P J, Evans K S, Carson G R and Marsh H C, Jr. (1992), “Cell surface expression of the C3b/C4b receptor (CR1) protects Chinese hamster ovary cells from lysis by human complement”, J Biol Chem 267: 24754-24761;

McDonald J F and Nelsestuen G L (1997), “Potent inhibition of terminal complement assembly by clusterin: Characterization of its impact on C9 polymerization”, Biochemistry 36: 7464-7473;

Medof M E, Kinoshita T and Nussenzweig V (1984), “Inhibition of complement activation on the surface of cells after incorporation of decay-accelerating factor (DAF) into their membranes”, J Exp Med 160: 1558-1578;

Moore M D, Cooper N R, Tack B F and Nemerow G R (1987), “Molecular cloning of the cDNA encoding the Epstein-Barr virus/C3d receptor (complement receptor type 2) of human B lymphocytes”, Proc Natl Acad Sci USA 84: 9194-9198;

Morgan E L (1986), “Modulation of the immune response by anaphylatoxins”, Complement 3: 128-136;

Mulligan M S, Schmid E, Beck-Schimmer B, Till G O, Friedl H P, Brauer R B, Hugli T E, Miyasaka M, Warner R L, Johnson K J and Ward P A (1996), “Requirement and role of C5a in acute lung inflammatory injury in rats”, J Clin Invest 98: 503-512;

Mulligan M S, Schmid E, Till G O, Hugli T E, Friedl H P, Roth R A and Ward P A (1997), “C5a-dependent up-regulation in vivo of lung vascular P-selectin”, J Immunol 158: 1857-1861;

Naniche D, Varior-Krishnan G, Cervoni F, Wild T F, Rossi B, Rabourdin-Combe C and Gerlier D (1993), “Human membrane cofactor protein (CD46) acts as a cellular receptor for measles virus”, J Virol 67: 6025-6032;

Nicholson-Weller A, Burge J, Fearon D T, Weller P F and Austen K F (1982), “Isolation of a human erythrocyte membrane glycoprotein with decay-accelerating activity for C3 convertases of the complement system”, J Immunol 129: 184-189;

Nicholson-Weller A and Wang C E (1994), “Structure and function of decay accelerating factor CD55”, J Lab Clin Med 123: 485-491;

Ogata R T, Mathias P, Bradt B M and Cooper N R (1993), “Murine C4b-binding protein: Mapping of the ligand binding site and the N-terminus of the pre-protein”, J Immunol 150: 2273-2280;

Okada N, Liszewski M K, Atkinson J P and Caparon M (1995), “Membrane cofactor protein (CD46) is a keratinocyte receptor for the M protein of the group A streptococcus”, Proc Natl Acad Sci USA 92: 2489-2493;

Parker C J (1992), “Regulation of complement by membrane proteins: An overview”, Curr Top Microbiol Immunol 178: 1-7;

Podack E R and MUller-Eberhard H J (1979), “Isolation of human S-protein, an inhibitor of the membrane attack complex of complement”, J Biol Chem 254: 9908-9914;

Ripoche J, Day A J, Harris T J and Sim R B (1988), “The complete amino acid sequence of human complement factor H”, Biochem J 249: 593-602;

Rollins S A, Zhao J, Ninomiya H and Sims P J (1991), “Inhibition of homologous complement by CD59 is mediated by a species-selective recognition conferred through binding to C8 within C5b-8 or C9 within C5b-9”, J Immunol 146: 2345-2351;

Rosenberg M E and Silkensen J (1995), “Clusterin: Physiologic and pathophysiologic considerations”, Int J Biochem Cell Biol 27: 633-645;

Schmid E, Piccolo M-T S, Friedl H P, Warner R L, Mulligan M S, Hugli T E, Till G O and Ward P A (1997a), “Requirement for C5a in lung vascular injury following thermal trauma to rat skin”, Shock 8: 119-124;

Seya T, Turner J R and Atkinson J P (1986), “Purification and characterization of a membrane protein (gp45-70) that is a cofactor for cleavage of C3b and C4b”, J Exp Med 163: 837-855;

Sharma A K and Pangburn M K (1996), “Identification of three physically and functionally distinct binding sites for C3b in human complement factor H by deletion mutagenesis”, Proc Natl Acad Sci USA 93: 10996-11001;

Spitzer R E, Vallota E H, Forristal J, Sudora E, Stitzel A, Davis N C and West C D (1969), “Serum C'3 lytic system in patients with glomerulonephritis”, Science 164: 436-437;

Sugita Y, Nakano Y, Oda E, Noda K, Tobe T, Miura N H and Tomita M (1993), “Determination of carboxyl-terminal residue and disulfide bonds of MACIF (CD59): A glycosyl-phosphatidylinositol-anchored membrane protein”, J Biochem 114: 473-477;

Sugita Y, Tobe T, Oda E, Tomita M, Yasukawa K, Yamaji N, Takemoto T, Furuichi K, Takayama M and Yano S (1989), “Molecular cloning and characterization of MACIF: An inhibitor of membrane channel formation of complement”, J Biochem 106: 555-557;

Suzuki S, Oldberg A, Hayman E G, Pierschbacher M D and Ruoslahti E (1985), “Complete amino acid sequence of human vitronectin deduced from cDNA: Similarity of cell attachment sites in vitronectin and fibronectin”, EMBO J 4: 2519-2524;

Tschopp J, Masson D, Schafer S, Peitsch M and Preissner K T (1988), “The heparin binding domain of S-protein/vitronectin binds to complement components C7, C8 and C9 and perform from cytolytic T-cells and inhibits their lytic activities”, Biochemistry 27: 4103-4109;

Vyse T J, Morley B J, Bartok I, Theodoridis E L, Davies K A, Webster A D and Walport M J (1996), “The molecular basis of hereditary complement factor I deficiency”, J Clin Invest 97: 925-933;

Weiler J M, Daha M R, Austen K F and Fearon D T (1976), “Control of the amplification convertase of complement by the plasma protein beta1H”, Proc Natl Acad Sci USA 73: 3268-3272;

Whaley K and Ruddy S (1976), “Modulation of C3b hemolytic activity by a plasma protein distinct from C3b inactivator”, Science 193: 1011-1013;

Yu J H, Abagyan R, Dong S H, Gilbert A, Nussenzweig V and Tomlinson S (1997), “Mapping the active site of CD59”, J Exp Med 185: 745-753;

Zhou Q S, Zhao J, Husler T, and Sims P J (1996), “Expression of recombinant CD59 with an N-terminal peptide epitope facilitates analysis of residues contributing to its complement-inhibitory function”, Mol Immunol 33: 1127-1134.

### B. Complement-Fixing Antibodies

By definition, a Complement-fixing antibody is an immunologically specific protein that combines with an antigen, or hapten, or epitope appearing on the exposed surface of a living cell, with such binding then leading to activation of Complement proteins, and then cell disruption or lysis in-situ.

The preferred Complement-fixing antibody will demonstrate at least two characteristics: It will have the capability of binding specifically to one or more epitopes present within a spatially exposed region of the breast epithelial cells then forming the cellular lining of the duct or are present as exfoliated cells within the lumen of the duct in vivo. In addition, the other essential attribute of the specific antibody is that, after binding to one or more epitopes present within a spatially exposed region of the epithelial cells, the bound antibody has the capacity to activate the cascade of Complement protein reactions, fix Complement, and cause disruption of cellular membranes and/or cell lysis in-situ. Both capabilities are necessary and required.

### Particular Antigens, Hastens, and Epitopes

The antigenic specificities and determinants of the Complement-fixing antibodies employed (singly or in combination) in this treatment method as immunological antagonists are quite diverse. For this reason, the useful range of antigens, haptens and epitopes will vary broadly and differ purposely with the particulars of the suspected breast carcinoma types and/or a prechosen range of specific surface determinants which are frequently found to appear of the exposed surfaces of normal, atypical and malignant duct epithelial cells in-vivo. The chosen Complement-fixing antibodies are specific for and will become immunologically bound in-situ to a wide variety of determinants which include and are presented by: a variety of different cytokeratins (CKs); various kinds of myoepithelial cells (MECs), epithelial cells, and basal cells; as well as cell type specific markers such as smooth muscle actin, calponin, p63, ER, PR, HER2/neu, CEA, CD10, epithelial membrane antigen (EMA) and vimentin.

### Kinds and Purity of Complement-Fixing Antibodies

The Complement-fixing antibodies employed as an immunological antagonist in the instant treatment method may be monoclonal, polyclonal, or synthetically fabricated using conventionally known manufacturing processes; can be prepared as human or humanized antibodies; can exist in any known type or format including IgG, IgM, and IgA; and can include the IgD, and IgE forms since hybrid molecules may be usefully employed. Note also that these different types of antibodies be employed singly, or in combined admixtures of any ratio or proportion.

In general therefore, the user has the option to choose whether the Complement-fixing antibody antagonist(s) is obtained from monoclonal, or polyclonal or broad antisera sources. Equally important, the user will decide whether the antibody or antibody fragments should be isolated and purified prior to use; whether they should be altered into humanized antibody form; or whether the antibody antagonist can be employed as a heterogeneous mixture of different entities and varying binding affinities, only some of which will have the requisite affinity and specific binding capability for an exposed epitope on the surface of an epithelial cell present within the ducts of the human breast. Thus, the degree of homogeneity, purity, human compatibility, affinity, and specificity of antibodies or antibody fragments and genetically engineered subunits for one or more antigens, haptens, or epitopes is left to the discretion and needs of the user.

### The Nature and Format of the Complement-Fixing Antibody Antagonist

The antigenic determinants recognized by the Complement-fixing antibodies are provided by the choice of antigens, haptens or epitopes used as immunogens; and by the manner in which the raising and harvesting of specific antibodies produced in response to the chosen immunogen(s) is made. However, this specific binding capability to a particular antigen, hapten, or epitope can be demonstrated not only by a whole intact antibody, but also by F(ab′)₂ fragments, as well as by Fab fragments derived from the whole antibody structure.

It will be recalled that while the whole IgG antibody molecule is a large bulky protein having two specific binding sites, the F(ab′)₂ fragment represents a divalent binding fragment of the whole antibody; while the Fab binding portion is a univalent binding unit having a minimum of antibody structure. In addition, similar smaller sized and genetically engineered antibody units having a specific binding capability have also been recently developed. Accordingly, all these specifically-binding antagonistic entities are deemed to be equally suitable for use in the present invention.

In addition, particular methods for preparing “humanized” antibodies have been devised. See for example, Co, M. S. and C. Queen, Nature 351: 501-502 (1991); Winter, G. and W. J. Harris, TiPs 14:139-142 (1993); Stephens et al., Immunology 85: 668-674 (1995); Kaku et al., Eur. J. Pharmacol. 279: 115-121 (1995); and the references cited within each of these publications. Humanized antibodies offer distinct therapeutic advantages; and thus are highly preferred for clinical use because they are less likely to provoke an undesired response from the patient undergoing treatment.

Other methods for preparing, isolating, and purifying each of these different antibody binding segments and units are conventionally known in the scientific literature and these techniques have been available for many years as common knowledge in this field. The user may thus chose from among all of these different structured formats—whole antibodies, antibody subunits and antibody fragments—in picking a useful antagonistic structure having a specific binding capability for an epitope in one of the spatially exposed regions of the epithelial cells existing within the ducts of the human breast.

### Immunogens

Any of the antigens and markers commonly appearing on the surface of an atypical, or pre-cancerous, or abnormal duct epithelial cell; or different fragments thereof; can potentially serve as an immunogen. Thus the expected range and intended variety of useful immunogens will typically include: any known type of cytokeratins (CKs); any known kind of myoepithelial cells (MECs), epithelial cells, and/or basal cells; and any cell type specific marker such as smooth muscle actin, calponin, p63, ER, PR, HER2/neu, CEA, CD10, epithelial membrane antigen (EMA) and vimentin. It is also expected and intended that the antibodies obtained with such immunogens will be empirically evaluated and chosen for use based upon their specific binding capacities and their Complement fixing properties.

It will be noted and appreciated also that the range and variety of the epitope binding demonstrated by the antibodies raised in response to these immunogens can vary widely and will typically provide a large number of different antigenic determinants. For example, if one chooses to use a peptide fragment as an immunogen, it will be recalled that a minimum of 5-7 amino acid residues (in theory) are able to be employed as a hapten in order to raise specific antibodies within a living host animal. However, longer peptide lengths of at least 10-20 residues are generally preferred because the raised specific antibodies will then show a much broader range of binding to vastly different determinants. Thus, if a great number of amino acid residues were purposely employed as the immunogen, a much larger number of different antigenic determinants becomes available, given the greater range of residue choices. Accordingly, the number of potential epitopes becomes enormous; yet each of these epitopes is a potential specific binding site for the antibody antagonist(s).

For these reasons, it is intended and envisioned that at least one peptide of suitable size (preferably at least 10-20 residues) be chosen as the immunogen in order to provide the antigenic determinants and cause the production of specific antibodies in a living host animal. However, after the amino acid residue size and composition has been chosen (in conformity with the requirement of being representative of a breast epithelial cell), the chosen antigen or hapten must be prepared or isolated as a chemical composition.

For this purpose, the desired amino acid sequence can be often synthetically prepared using conventionally known solid phase peptide synthesis methods [such as Merrifield, R B, J. Am. Chem. Soc. 85: 2149 (1963)]. If synthesized, it is most desirable that the chosen peptide be purified (such as by gel filtration) and desirably analyzed for content and purity (such as by sequence analysis and/or mass spectroscopy).

After its isolation or synthesis, the chosen peptide composition is typically coupled to a carrier to form the immunogen as a whole. Suitable carriers available for this purpose are conventionally available in a great variety from many diverse sources. The only requirements regarding the characteristics and properties of the carrier are: first, that the carrier be in fact antigenic alone or in combination with the chosen amino acid residue sequence; and second, that the carrier be able to present the antigenic determinants of the residue sequence such that antibodies specific against the amino acid residues are produced in a living host animal.

The preferred choices of a carrier suitable for immunization purposes today include keyhold limpet hemocyanin (KLH), coupled by glutaraldehyde (GLDH), sulfo-m-maleimidobenzo (M-hydroxysuccinimide) ester (MBS), or bisdiazobenzidine (BDB). However, any other carrier compatible with the host to be immunized is also suitable for use. Example of such other carriers include bovine serum albumin, thyroglobulin, and the like.

### Immunization Procedure

All immunizations and immunization procedures are performed in the conventionally known manner described in the scientific literature. It is expected that under certain use conditions, adjuvants will be employed in combination with the prepared immunogen(s). Alternatively, the prepared immunogen(s) may be used alone and be administered to the animal or human host in any manner which will initiate the production of specific antibodies.

In addition, the harvesting of polyclonal antiserum and the isolation of antibody containing sera or antibody producing cells follows the conventionally known techniques and processes for this purpose. Similarly, the preparation of hybridomas follows the best practices developed over recent years for the isolation of monoclonal antibodies [Marshak-Rothstein et al., J. Immunol. 122: 2491 (1979)].

Once obtained, the polyclonal antisera and/or monoclonal antibodies and/or genetically engineered antibodies should be evaluated and verified for their ability to bind specifically with an epitope existing within a spatially exposed region of a duct epithelial cell, as well as for the capability to fix Complement and initiate the cascade of Complement reactions which result in breast epithelial cell disruption and/or lysis. If desired, cleavage of the raised antibodies with papain will produce two Fab fragments plus the Fc fragment; whereas cleavage of the antibodies with pepsin produces the divalent F(ab′)₂ fragment and the Fc′ fragment—all as conventionally known.

It will be expressly understood, however, that regardless of whether the antibody binding portion represents polyclonal antisera, monoclonal antibodies, the F(ab′)₂ fragment, Fab fragments, humanized antibodies, or any other antibody species—all of these formats are suitable and intended for use so long as the Complement-fixing capability is demonstrated after binding to at least one epitope existing on the surface of breast epithelial cells in-vivo.

It is therefore necessary and expected that a wide variety of different immunoassay systems will be employed to demonstrate the specific binding and Complement-fixing capabilities required by the antibody antagonists of the present invention; and that the parameters of concentration, volume, temperature, carriers, and delivery systems can be varied extensively at will when choosing antibodies and/or antibody fragments and subunits. The present invention therefore presumes and incorporates by reference any conventionally known immunoassay technique, procedure, protocol, or other factor or parameter—all of which may be usefully employed for the evaluation and/or preparation of a specifically binding and Complement-fixing antibody antagonist.

### Background Scientific Publications

For overall knowledge and general benefit, a summary listing of some scientific publications concerned with antibodies is presented here. All of these scientific publications, however, are deemed to be of merely background interest and limited informational value. These scientific publications include:

Adkins et al. (1998), “Edrecolomab (Monoclonal Antibody 17-1A)”, Drugs 56(4):619-626;

Anderson et al. (1980), “Characterization of the Fc Receptor for IgG on a Human Macrophage Cell Line U937”, I. Immunol. 125(6):2735-41;

Angal et al. (1993), “A Single Amino Acid Substitution Abolishes the Heterogeneity of Chimeric Mouse/Human (IgG4) Antibody”, Molecular Immunology 30(1):105-108;

Batova et al. (1999), “The Ch 14.18-GM-CSF Fusion Protein is Effective at Mediating Antibody-Dependent Cellular Cytotoxicity and Complement-Dependent Cytotoxicity in Vitro”, Clinical Cancer Research 5:4259-4263;

Batra et al. (1993), “Insertion of Constant Region Domains of Human IgG1 into CD4-PE40 Increases Its Plasma Half-Life”, Molecular Immunology 30(4) :379-386;

Bjorn et al. (1985), “Evaluation of Monoclonal Antibodies for the Development of Breast Cancer Immunotoxins,” Cancer Research, 45:1214-1221; Boulianne et al., (1984), “Production of Functional Chimaeric Mouse/Human Antibody”, Nature 312:643-6;

Bourgois et al. (1974), “Determination of the Primary Structure of a Mouse IgG2a Immunoglobulin: Amino-Acid Sequence of the Fc Fragment: Implications for the Evolution of Immunoglobulin Structure and Function”, Eur. J. Biochem. 43:423-35;

Brambell et al. (1964), “A Theoretical Model of Gamma-Globulin Catabolism”, Nature 203:1352-55;

Brekke et al. (1994), “Human IgG Isotype-Specific Amino Acid Residues Affecting Complement-Mediated Cell Lysis and Phagocytosis”, Eur. J. Immunol. 24:2542-2547;

Canfield et al. (1991), “The Binding Affinity of Human IgG for its High Affinity Fc Receptor is Determined by Multiple Amino Acids in the CH2 Domain and is Modulated by the Hinge Region”, J. Exp. Med. 173(6):1483-1491;

Capon et al., (1989), “Designing CD4 Immunoadhesins for AIDS Therapy,” Nature 337:525-531;

Caton et al. (1986), “Structural and Functional Implications of a Restricted Antibody Response to a Defined Antigenic Region on the Influenza Virus Hemagglutinin”, The EMBO Journal 5(7):1577-1587;

Chapman et al. (1994), “Mapping Effector Functions of a Monoclonal Antibody to GD3 by Characterization of a Mouse-Human Chimeric Antibody”, Cancer Immuno. Immunother. 39:198-204;

Chappel et al. (1991), “Identification of the Fc Gamma Receptor Class I Binding Site In Human IgG Through Use of Recombinant IgG1/IgG2 Hybrid and Point-Mutated Antibodies,” Proc. Natl. Acad. Sci. USA 88:(20):9036-40;

Chaudhary et al. (1989), “A Recombinant Immunotoxin Consisting of Two Antibody Variable Domains Fused to Pseudomonas Exotoxin”, Nature 339:394-397;

Cheon et al. (1994), “High-Affinity Binding Sites for Related Fibroblast Growth Factor Ligands Reside Within Different Receptor Immunoglobulin-Like Domains”, Proc. Natl. Acad. Sci. USA 91: 989-993;

Cole et al. (1997), “Human IgG2 Variants of Chimeric Anti-CD3 Are Nonmitogenic to T Cells,” Journal of Immunology, 159:3613-3621; Dorai et al., (1991), “Aglycosylated Chimeric Mouse/Human IgG1 Antibody Retains Some Effector Function”, Hybridoma 10(2):211-217;

Dorai et al. (1992), “Role of Inter-Heavy and Light Chain Disulfide Bonds in the Effector Functions of Human IgG1”, Molecular Immunology 29(12): 1487-1491;

Duncan et al. (1988), “The Binding Site for C1q on IgG,” Nature, 332:738-740; Ellison et al., (1982), “The Nucleotide Sequence of a Human Immunoglobulin C Gamma₁ Gene”, Nucleic Acids Res. 10:4071-9;

Fell et al. (1991), “Genetic Construction and Characterization of a Fusion Protein Consisting of a Chimeric F(ab′) with Specificity for Carcinomas and Human IL-2”, J. Immunology 146(7):2446-2452;

Fell et al. (1992), “Chimeric L6 Anti-Tumor Antibody: Genomic Construction, Expression, and Characterization of the Antigen Binding Site,” J. Biological Chemistry, 267:15552-15558;

Gillies et al. (1989), “High-Level Expression of Chimeric Antibodies Using Adapted cDNA Variable Region Cassettes”, J. Immunol. Methods 125:191-202;

Gillies et al. (1990), “Antigen Binding and Biological Activities of Engineered Mutant Chimeric Antibodies with Human Tumor Specificities”, Hum. Antibod. Hybridomas 1(1):47-54;

Gillies et al. (1991), “Expression of Genetically Engineered Immunoconjugates of Lymphotoxin and a Chimeric Anti-Ganglioside GD2 Antibody”, Hybridoma 10(3):347-56;

Gillies et al. (1993), “Biological Activity and In Vivo Clearance of Antitumor Antibody/Cytokine Fusion Proteins”, Bioconjugate Chem. 4(3):230-235;

Gillies et al. (1998), “Antibody-IL-12 Fusion Proteins are Effective in SCID Mouse Models of Prostate and Colon Carcinoma Metastases”, J. Immunology 160:6195-6203;

Guyre et al. (1997), “Increased Potency of Fc-Receptor-Targeted Antigens”, CancerImmunol. Immunother. 45:146-148;

Harvill et al. (1995), “An IgG3-IL2 Fusion Protein Activates Complement, Binds Fc.gamma.RI, Generates LAK Activity and Shows Enhanced Binding to the High Affinity IL-2R”, Immunotechnology 1:95-105;

He et al. (1998), “Humanization and Pharmacokinetics of a Monoclonal Antibody with Specificity for Both E-and P-Selectin”, J. Immunology 160:1029-1035;

Hellstrom et al. (1986), “Antitumor Effects of L6, and IgG2a Antibody that Reacts with Most Human Carcinomas”, Proc. Natl. Acad. Sci. USA 83: 7059-7063;

Hulett et al. (1994), “Molecular Basis of Fc Receptor Function”, Adv. Immunol. 57:1127;

Hurn et al. (1980), “Production of Reagent Antibodies”, Methods in Enzymology 70: 104-142;

Huston et al. (1988), “Protein Engineering of Antibody Binding Sites: Recovery of Specific Activity in an Anti-Digoxin Single-Chain Fv Analogue Produced In Escherichia coli”, Proc. Natl. Acad. Sci. USA 85:5879-5883;

Isaacs et al. (1998), “Therapy with Monoclonal Antibodies. II. The Contribution of Fc.gamma. Receptor Binding and the Influence of CH1 and CH3 Domains on In Vivo Effector Funcion”, J. Immunol. 161:3862-3869;

Isenman et al. (1975), “The Structure and Function of Immunoglobulin Domains: II. The Importance of Interchain Disulfide Bonds and the Possible Role of Molecular Flexibility in the Interaction between Immunoglobulin G and Complement”, J. Immunology 114(6):1726-1729;

Jefferis et al. (1990), “Molecular Definition of Interaction Sites on Human IgG for Fc Receptors huFc.gamma.R”, Mol. Immunol. 27(12):1237-1240;

Jones et al., (1986), “Replacing the Complementarity-Determining Regions in a Human Antibody with Those from a Mouse,” Nature, 321:522-525;

Karpovsky et al. (1984), “Production of Target-Specific Effector Cells using Hetero-Cross Linked Aggregate Containing Anti-Target Cell and AntiFc.gamma. Receptor Antibodies”, Journal of Experimental Medicine 1609(6): 1686-1701;

Lo et al. (1992), “Expression and Secretion of an Assembled Tetrameric CH2-Deleted Antibody in E. coli.” Hum. Antibod Hybridomas 3:123-128;

Lo et al. (1998), “High Level Expression and Secretion of Fc-X Fusion Proteins in Mammalian Cells”, Protein Engineering 11(6):495-500;

Martin et al. (2001), “Crystal Structure at 2.8 ANG of an FcRn/Heterodimeric Fc Complex: Mechanism of pH-Dependent Binding”, Mol. Cell. 7(4):867-77;

Menard et al. (1983), “Generation of Monoclonal Antibodies Reacting with Normal and Cancer Cells of Human Breast”, Cancer Res. 43:1295-300;

Morrison et al. (1984), “Chimeric Human Antibody Molecules: Mouse Antigen-Binding Domains with Human Constant Region Domains”, Proc. Natl. Acad. Sci. USA 81:6851-5;

Mueller et al. (1990), “Enhancement of Antibody-Dependent Cytotoxicity With A Chimeric Anti-GD2 Antibody”, J. Immunology 144(4): 1382-1386;

Mueller et al. (1997), “Humanized Porcine VCAM-Specific Monoclonal Antibodies with Chimeric IgG2/G4 Constant Regions Block Human Leukocyte Binding to Porcine Endothelial Cells”, Molecular Immunology 34(6):441-452;

Neuberger et al. (1984), “Recombinant Antibodies Possessing Novel Effector Functions”, Nature 312:604-608;

Ngo et al. (1994), “Computational Complexity, Protein Structure Prediction, and the Levinthal Paradox”, in The Protein Folding Problem and Tertiary Structure Prediction, Merz et al. (eds.), pp. 433-440 and 492-495, Birkhauser, Boston, Mass.;

Poon et al. (1995), “Structure and Function of Several Anti-Dansyl Chimeric Antibodies Formed by Domain Interchanges Between Human IgM and Mouse IgG2b”, J. Biol. Chem. 270:8571-7;

Reisfeld et al. (1994), “Potential of Genetically Engineered Anti-Ganglioside GD2 Antibodies for Cancer Immunotherapy”, Prog. Brain Res. 101:201-212;

Riechmann et al. (1988), “Reshaping Human Antibodies for Therapy”, Nature 332:323-7;

Schlom (1991), “Monoclonal Antibodies: They're More and Less Than You Think”, in Molecular Foundations of Oncology, pp. 95-133;

Takai (2002), “Roles of Fc Receptors in Autoimmunity”, Nat. Rev. Immunol. 2(8):580-92;

Tao et al. (1989), “Studies of Aglycosylated Chimeric Mouse IgG: Role of Carbohydrate in the Structure and Effector Functions Mediated by the Human IgG Constant Region”, J. Immunology 143(8):2595-2601;

Tao et al. (1993), “Structural Features of Human Immunoglobulin G that Determine Isotype-Differences in Complement Activation,” J. Exp. Med. 178(2):661-667.

Thommesen et al. (2000), “Lysine 322 in the Human IgG3 CH2 Domain is Crucial for Antibody Dependent Complement Activation”, Mol. Immunol. 37(16):995-1004;

Ward et al.,(1995), “The Effector Functions of Immunoglobulins: Implications for Therapy”, Therapeutic. Immunology 2:77-94;

Went et al. (2004), “Frequent EpCam Protein Expression in Human Carcinomas”, Human Pathology 35(1): 122-128;

Woof et al. (1986), “Localisation of the Monocyte-Binding Region on Human Immunoglobulin G”, Mol. Immunol. 23:319-30;

Xu et al. (1994), “Residue at Position 331 in the IgG1 and IgG4 CH2 Domains Contributes to Their Differential Ability to Bind and Activate Complement”, J. Biol. Chem. 269(5):3469-3474.

### C. The Biocompatible Fluid Carrier

The essential purpose and function of the liquid solution or suspension fluid is to serve as a biocompatible carrier for at least one Complement-fixing antibody (specific for an antigen, hapten or epitope of normal, atypical or malignant duct/lobule epithelial cells) and the appropriate Complement reaction proteins which are to become activated and subsequently fixed in-situ by the then bound antibody. Accordingly, any aqueous based fluid which is demonstrably non-reactive, non-toxic, and effectively biochemically neutral in its in-vivo effects is suitable for use when preparing the carrier.

In addition, since the Complement-fixing antibody and the Complement reaction proteins are to be introduced via the carrier fluid into and through the ducts of the human breast—i.e., as an intrusive application, the appropriate quantities of antagonistic antibody and Complement reaction proteins should be prepared in sterile form; can be made in single or multiple dose formats; and will typically be dispersed in a sterile fluid carrier such as physiological saline, 5% dextrose solutions, serum, or plasma.

It is most desirable that the fluid carrier be biocompatible with the cells and tissues forming the ducts of the human breast. For this reason, physiological strength electrolytes are typically present in the carrier fluid. In addition, a broad range of other chemical agents and additives may be included as non-essential ingredients in formulations of the fluid carrier. These non-essential ingredients can be water soluble, water miscible, or aqueous suspensions; and will typically include various minerals and salts, lidocaine or other local anesthetics, one or more coloring agents, water soluble antimicrobial preservatives, and the like.

### IV. Dosages & Modes of Administration

Formulated compositions embodying the specifically binding Complement-fixing antibodies and the Complement reaction proteins can be administered in any manner which preserves the activity and functions of these entities and delivers them to the breast epithelial cells forming the cellular lining of the ducts or existing as exfoliated cells within the lumen of the ducts. The prepared antagonistic antibody and Complement reaction proteins can be introduced by any means or routing equipment that introduces these reactants into and through the ducts of the human breast as described above.

The dosage of the formulated treatment fluid to be administered to any living human patient will of course vary with and be dependent upon the age, overall health, and weight of the recipient; the kind of concurrent treatment, if any; the frequency of concurrent treatment; and the physician's current prognosis for the patient.

In general however, a range dose of antagonistic antibody from 0.1 milligrams to about 10.0 milligrams per kilogram of body weight, in twice weekly or three times weekly administrations is expected to be effective to yield the desired therapeutic result. The quantity of accompanying Complement reaction proteins will vary directly with the dose of antagonistic antibody employed in the treatment, but should always be enough to insure that there is a sufficient concentration of Complement reaction proteins for activation to occur and fixation to proceed to completion.

The duration of treatment with antagonistic antibody and Complement reaction proteins is expected to be continued so long as a favorable clinical result is obtained. It is believed that this treatment regimen will destroy atypical and malignant epithelial cells then present within the ducts of the human breast; will inhibit the growth of pre-cancerous cells in-vivo; and will also act to retard or halt the growth of breast carcinomas in-situ. However, it is as yet unclear whether or not this inhibitory treatment method will provide for complete regression of tumor. For this reason especially, the treatment duration and dosage should be monitored accordingly.

### V. The Outcome and Result of using the In-Vivo Treatment Method among the Very Desirable Outcomes and Consequences of using the Treatment Method of the Present Invention are those Identified Below

### In-Vivo Results:

1. The normal, atypical, and malignant epithelial cells that cause breast carcinoma would no longer be present in-vivo within the ducts/lobules of the patient. Instead, internally scarred but intact breasts remain.

2. Ductal carcinoma in-situ and lobular carcinoma in-situ—along with the ductal and lobular epithelium—would be selectively destroyed without damage or injury to any other system, organ, or tissue in the patient's body. Currently, radiation and major surgery is often employed as a treatment for those women patients having a clinical diagnosis of ductal carcinoma in-situ or lobular carcinoma in-situ, even though these types of carcinoma often do not develop into invasive tumors.

### Benefits and Advantages of the Treatment Method:

(i). The antibodies in the cocktail can be monoclonal, polyclonal or humanized.

(ii). Lidocaine or another local anesthetic can be part of the cocktail or it can be infused as a prior lavage step to control pain or discomfort.

(iii). Women with BRCA1 and BRCA2 genetic findings often undergo prophylactic bilateral mastectomies. This treatment would offer another option.

(iv). Men with the BRCA2 genetic findings have about the same breast carcinoma risk as women and could also benefit from such a treatment.

(v). This methodology may also be used as an immunological prevention technique as well as a therapeutic treatment procedure.

(vi). In contrast with the currently available breast carcinoma screening procedures and the alternative treatments of surgery, radiation, and chemotherapy, the present method offers multiple cosmetic and psychological advantages; less anesthesia risk; and less surgery risk. Mammography radiation also increases breast carcinoma risk and this risk can be avoided by using the present methodology.

(vii). Since one in eight women get breast carcinoma, the expected cost savings for treating breast carcinomas are large.

(viii). There are Complement inhibitors appearing on the surfaces of breast duct epithelial cells such as CD59, CD46, and CD55. These have been inactivated by the use of antibodies directed at them. Thus the use of such additional antibodies added to the above described cocktail or as an earlier performed lavage step (such as washing with lidocaine) may be employed.

(ix). Alternative kinds of Complement-fixing antibodies directed at Complement inhibitors can also be optionally added to the prepared fluid admixture.

(x). Human or animal derived serum or plasma can serve as a convenient source of Complement reaction proteins in a fluid carrier with electrolytes. Irradiation, filtration, or other purification means (instead of dry heat or steam heat), can be used to render the prepared formulation safe from microorganisms.

(xi). Bacteria can inactivate Complement and Complement reactive proteins. For this reason, mastitis and other infections of the breast should be successfully treated with antibiotics and the like before the ductal lavage treatment method is initiated. It is also preferable, in this instance, that multiple repeat ductal lavage treatments be performed after the bacterial infection is resolved.

(xii). Many subpopulations of breast cancer cells are known to be tumorigenic—i.e., these subpopulations of cells can themselves form new tumor growth in-vivo. These subpopulations of cells are today believed to have stem cell-like properties; and thus are very attractive as a specific antibody target. Typically, these subpopulations of cells have distinct phenotypes, as revealed by the presence of specific membrane antigens; and thus can be identified by showing the existence of particular markers on the cell surface such as CD44⁺ and CD24^{-/low}.

Accordingly, the present invention intends and expects that these cell markers/antigens can and shall be used as additional targets for reactive binding by specific Complement-fixing antibodies (which have been raised against and thus are specific for these CD44⁺ and CD24^{-/low} phenotypic markers); and is very desirable that such Complement-fixing antibodies specific for these CD44⁺ and CD24^{-/low} antigens be purposely added to the formulated ducted lavage treatment fluids intended for use using the present treatment method.

### VI. Representative Formulations of the Prepared Treatment Fluid

Merely to illustrate the broad range and variety of differing formulations which can be employed in the instant treatment method, several representative formulations are set forth below. It is expressly recognized, however, that the particular formulations given below are exemplary embodiments; none of which are limiting or restrictive of the possibilities that can be prepared and usefully employed.

### A Minimal Exemplary Formulation:

Monoclonal anti-epithelial membrane antibody, and

Human serum (irradiated).

### A Middle Range Exemplary Formulation:

Lidocaine (1%),

Monoclonal anti-epithelial membrane antibody,

Human serum (irradiated),

Anti-p63 antibody, and

Anti-CEA

### A Preferred Exemplary Formulation:

Lidocaine (1%),

Monoclonal anti-epithelial membrane antibody,

Human serum (irradiated),

Anti-p63 antibody,

Anti-CD59 antibody, and

Anti-CD44 antibody.

### VII. An Exemplary Protocol

As an aid and useful guide for the clinician, a medical protocol is set forth below which is deemed to be an exemplary recitation of a clinical procedure for practicing the present treatment methodology. It will be expressly understood, however, that the following recitation is merely illustrative and representative of how the treatment method as a whole may be performed; and this particular example is neither limiting nor restrictive of the many other possible formats and manners for practicing the present invention.

### An Exemplary Protocol:

In this illustrative instance, the modified ductal lavage treatment method is intended to be performed in a typical doctor's office as an outpatient clinical procedure. However, it is expected that a two or three person team will be needed to perform the protocol with a minimum of difficulty and time.

Initially, 1% lidocaine is applied to the nipples of the patient up to one hour's time before performing the treatment protocol. Breast massage and heat packs may be applied to the breasts to encourage fluid production.

A 10 cc sterile syringe with tubing to a clear cap is then placed over the nipples. Negative pressure (vacuum) is applied to identify the duct openings as fluid is expressed out of them. Duct dilators may be used to open the ducts further.

A second sterile syringe is desirably used in combination with a microcatheter to apply 1% lidocaine as a local anesthetic into each open duct. After sufficient time for local anesthesia to occur has passed, another sterile syringe containing the formulated treatment fluid of choice [i.e., the prepared fluid mixture of specific antibody or antibodies, Complement proteins, and liquid carrier] is introduced into each duct via the microcatheter, and is used to infuse each duct with about 8-10 cc's of the formulated treatment fluid. The complete filing of the individual ducts with treatment fluid is indicated when fluid starts to exist out of the duct onto the nipple.

Subsequently, after about 5-10 minutes reaction time (or such reaction time as is deemed sufficient by the attending physician), the fluid in each duct lumen can be then withdrawn and extracted, preferably using a different syringe for this purpose. The initial treatment is then complete.

Nevertheless, it is preferable that multiple treatments of the ducts be made as part of the overall protocol. Accordingly, a second treatment of the ducts in each breast should then be made using either the same formulated fluid as in the initial treatment or a differently formulated treatment fluid [i.e., another formulation using different specific antibody or antibodies, Complement proteins, and liquid carrier in fluid admixture] be performed for the patient. This second treatment would cyclically repeat the introduction of treatment fluid into each open duct; allow sufficient time for the introduced treatment fluid to react within each open duct; and then be extracted and removed from each individual duct by syringe.

In some instances, even a third treatment of the ducts in each breast can and should be made. Here also, the formulated treatment fluid employed on the third occasion can be made using either the same formulated fluid as in the initial or second treatment or a markedly changed third formulated treatment fluid.

During the course of performing the protocol, sterile gauze may be used to minimize skin or nipple exposure to the chosen formulated treatment fluid(s). This should be done when treatment fluid is expressed into the individual ducts, and when the ducts become completely filled with treatment fluid and starts to exit out of the duct. The sterile gauze is used to soak up the excess fluid as it appears.

Similarly, petroleum jelly can be used to precoat and protect the skin and nipples from accidental contact with the formulated treatment fluid. The petroleum jelly can be used to surround and not cover areas of Paget's disease of breast, of the nipple, areola and skin. Paget's disease of the breast occurs in 1% to 2% of female patients and in less than 5% of men with mammary carcinoma. Most of these women have a clinically evident nipple lesion.

Sterile gloves should be worn by the person performing the technique for the patient; and the breasts of the patient can be disinfected both before and after the treatment protocol has been performed. The nipples may optionally also be covered with a sterile bandage at the conclusion of the treatment.

The preset invention is not restricted in form nor limited in scope, except by the claims appended hereto.

What I claim is:

1. A method for therapeutically treating a living human patient believed to be afflicted with an identifiable type of atypical or abnormal epithelial cell in the breast, said method comprising the steps of:
identifying at least one duct in the nipple of the human breast as a target for therapeutic treatment;
introducing a predetermined quantity of a formulated fluid into the lumen of said identified duct as a therapeutic treatment, wherein said introduced treatment fluid comprises
(i) at least one Complement-fixing antibody directed against and able to bind to specific antigens, haptens, or epitopes which are characteristically present on normal, atypical, or malignant breast epithelial cells in-vivo,
(ii) Complement proteins forming the chemical components of Complement and sufficient for initiating the cascade of reactions for in-vivo activation and fixation of Complement by said antibody after becoming bound to a breast epithelial cell, and
(iii) a biocompatible fluid carrier;
allowing said introduced treatment fluid to react in-situ for a preselected time period with such breast epithelial cells as are then present within the lumen of said identified duct, whereby at least a portion of said antibodies in said introduced treatment fluid become bound to the breast epithelial cells, and there is an in-situ activation and fixing of Complement proteins by said antibodies after becoming bound to the breast epithelial cells; and
removing said reacted treatment fluid from the lumen of said identified duct in the human breast.

2. A method for prophylactically treating a living human patient suspected of becoming afflicted with an identifiable type of atypical or abnormal epithelial cell in the breast, said method comprising the steps of:
identifying at least one duct in the nipple of the human breast as a target for prophylactic treatment;
introducing a predetermined quantity of a formulated fluid into the lumen of said identified duct as a prophylactic treatment, wherein said introduced treatment fluid comprises
(i) at least one Complement-fixing antibody directed against and able to bind to specific antigens, haptens, or epitopes which are characteristically present on normal, atypical, or malignant breast epithelial cells in-vivo,
(ii) Complement proteins forming the chemical components of Complement and sufficient for initiating the cascade of reactions for in-vivo activation and fixation of Complement by said antibody after becoming bound to a breast epithelial cell, and
(iii) a biocompatible fluid carrier;
allowing said introduced treatment fluid to react in-situ for a preselected time period with such breast epithelial cells as are then present within the lumen of said identified duct, whereby at least a portion of said antibodies in said introduced treatment fluid become bound to the breast epithelial cells, and there is an in-situ activation and fixing of Complement proteins by said antibodies after becoming bound to the breast epithelial cells; and
removing said reacted treatment fluid from the lumen of said identified duct in the human breast.

3. The treatment method as recited in claim 1 or 2 wherein said whereby said treatment method produces at least one result selected from the group consisting of
cell membrane disruption in-vivo for at least some epithelial cells then present within said identified duct,
cell lysis in-vivo for at least some epithelial cells then present within said identified duct,
an in-vivo denuding of at least some normal, atypical and abnormal cells from the surface of the epithelium of said identified duct, and
scarring and normal cell repair for the denuded surface then present within said identified duct.

4. The treatment method as recited in claim 1 wherein said treatment is employed against an abnormal cell present within a neoplasm selected from the group consisting of benign breast tumors, ductal carcinomas, medullary carcinomas, and lobar carcinomas.

5. The treatment method as recited in claim 2 wherein said treatment is employed against an atypical cell present within a precursor lesion selected from the group consisting of precursor cellular lesions of benign breast tumors, precursor cellular lesions of ductal carcinomas, and precursor cellular lesions of lobular carcinomas, precursor cellular lesions of medullary carcinomas, and precursor cellular lesions of other malignancies.

6. The treatment method as recited in claim 1 or 2 wherein said Complement-fixing antibody of said introduced treatment fluid is selected from the group consisting of IgG, IgM, IgA, IgD, and IgE antibodies.

7. The treatment method as recited in claim 1 or 2 wherein said Complement-fixing antibody of said introduced treatment fluid is selected from the group consisting of human and humanized antibodies.

8. The treatment method as recited in claim 1 or 2 wherein said Complement-fixing antibody of said introduced treatment fluid comprises multiple types of antibodies in combined admixture.

9. The treatment method as recited in claim 1 or 2 wherein said Complement-fixing antibody of said introduced treatment fluid is selected from the group consisting of by F(ab′)₂ fragments and Fab fragments derived from the whole antibody structure.

10. The treatment method as recited in claim 1 or 2 wherein said Complement-fixing antibody of said introduced treatment fluid is specific for and will become immunologically bound in-situ to at least one selected from the group consisting of basal cells, luminal cells, epithelial cells, and myoepithelial cells.

11. The treatment method as recited in claim 1 or 2 wherein said Complement-fixing antibody of said introduced treatment fluid is specific for and will become immunologically bound in-situ to at least one entity selected from the group consisting of a cytokeratin, smooth muscle actin, calponin, p63, ER, PR, HER2/neu, CEA, CD10, epithelial membrane antigen, and vimentin.

12. The treatment method as recited in claim 1 or 2 wherein said Complement reaction proteins of said introduced treatment fluid are activated via the classical pathway.

13. The treatment method as recited in claim 1 or 2 wherein said Complement reaction proteins of said introduced treatment fluid are activated via the classical pathway of reactions.

14. The treatment method as recited in claim 1 or 2 wherein said Complement reaction proteins of said introduced treatment fluid are activated via the alternative pathway of reactions.

15. The treatment method as recited in claim 1 or 2 wherein said biocompatible fluid carrier of said introduced treatment fluid is selected from the group consisting of physiological saline, 5% dextrose solutions, serum, and plasma.

16. The treatment method as recited in claim 1 or 2 wherein said biocompatible fluid carrier of said introduced treatment fluid includes at least one entity selected from the group consisting of minerals, salts, an anesthetic, a coloring agent, and an antimicrobial preservative.
